(19) United States
(12) Patent Application Publication
(10) Pub. No.: US 2021/0340599 A1
(43) Pub. Date: Nov. 4, 2021

(54) PREDICTING ANTIBIOTIC RESISTANCE AND COMPLEMENTARY ANTIBIOTIC COMBINATIONS

(71) Applicant: International Business Machines Corporation, Armonk, NY (US)

(72) Inventors: James Kaufman, San Jose, CA (US); Ed Seabolt, Williamson, TX (US); Kristen Beck, San Jose, CA (US); Mary Ann Roth, San Jose, CA (US); Akshay Agarwal, Sunnyvale, CA (US); Gowri Nayar, San Jose, CA (US)

(21) Appl. No.: 16/865,743

(22) Filed: May 4, 2020

(51) Int. Cl. C12Q 1/689; G16B 5/00; G16B 40/00; G16B 50/30

(52) U.S. Cl. CPC C12Q 1/689; G16B 5/00; C12Q 2600/106; G16B 50/30; G16B 40/00

(57) ABSTRACT

Techniques are provided for predicting antibiotic resistance from functional omics data and recommending complementary combinations of antibiotics. According to an embodiment, computer implemented method can comprise identifying, by a system operatively coupled to at least one processor, one or more proteins that have one or more functional domains associated with at least one code selected from a coding system for a set of phenotypes, and modelling, by the system, the one or more proteins as a functional capacity vector. In some implementations, the method can further include selecting the coding system and/or the at least one code based on a phenotype of interest. The method can further comprise employing, by the system, the functional capacity vector to identify one or more antibiotic compounds to which an organism within the set of phenotypes is resistant or susceptible, and/or to predict complementary antibiotic combinations.

### TECHNICAL FIELD

This application relates to techniques for predicting antibiotic resistance from functional omics data and recommending complementary combinations of antibiotics.

### SUMMARY

The following presents a summary to provide a basic understanding of one or more embodiments of the present disclosure. This summary is not intended to identify key or critical elements or to delineate any scope of the particular embodiments or any scope of the claims. Its sole purpose is to present concepts in a simplified form as a prelude to the more detailed description that is presented later. In one or more embodiments described herein, devices, systems, computer-implemented methods, and/or computer program products are provided that can predict antibiotic resistance from functional omics data and recommend complementary combinations of antibiotics.

According to an embodiment, a system can comprise a memory that stores computer executable components and a processor that executes the computer executable components stored in the memory. The computer executable components comprise a protein identification component that identifies one or more proteins that have one or more functional domains associated with at least one code selected from a coding system for a set of phenotypes. The computer executable components further comprise a vectorization component that models the one or more proteins as a functional capacity vector. In various implementations, the computer executable components can also comprise a coding system selection component that that selects the coding system based on a phenotype of interest, and a code selection component that that selects the at least one code based on the phenotype of interest.

In one or more implementations, the computer executable components can also comprise a susceptibility forecasting component that employs the functional capacity vector to identify one or more antibiotic compounds to which an organism within the set of phenotypes is resistant. The susceptibility forecasting component can also employ the functional capacity vector to identify one or more antibiotic compounds to which an organism within the set of phenotypes is susceptible. In addition, in some implementations, the susceptibility forecasting component can employ the functional capacity vector to predict one or more minimum inhibitory concentrations for one or more antibiotic compounds against an organism included within the set of phenotypes. In one or more additional implementations, the computer executable components can also comprise a combination forecasting component that employs the functional capacity vector to identify one or more antibiotic compound combinations to which an organism within the set of phenotypes is susceptible.

According to another embodiment, a computer program product is provided for representing a genome with a dimensionally reduced coding vector that represents one or more target functions associated with the genome within a target phenotypic space. The computer program product comprising a computer readable storage medium having program instructions embodied therewith, wherein the program instructions are executable by a processing component to cause the processing component to identify one or more target genes of the genome that encode one or more proteins responsible for the one or more target functions, and generate a functional capacity vector for the genome using one or more distinct codes assigned to the one or more target functions.

In various implementations, the program instructions can further cause the processing component to select a coding system for a set of phenotypes included in the target phenotypic space, wherein the coding system identifies different functions observed for the set of phenotypes and assigns distinct codes to the different functions, and determine the one or more distinct codes using the coding system. The program instructions can also cause the processing component to determine one or more functional domains respectively associated with the one or more distinct codes, identify the one or more proteins based on the one or more proteins comprising the one or more functional domains, and generate the functional capacity vector based on the one or more proteins. In some implementations, the program instructions can further cause the processing component to employ the functional capacity vector to identify one or more antibiotic compounds to which an organism included within target phenotypic space is susceptible.

In one or more additional embodiments, another system is provided that a system can comprise a memory that stores computer executable components and a processor that executes the computer executable components stored in the memory. The computer executable components comprise a reference data generation component that generates a reference data structure identifying different genomes, antimicrobial resistance statuses of the different genomes to different antibiotic compounds, and functional capacity vectors for the different genomes, wherein the functional capacity vectors represent sets of phenotypic features expressed by the different genomes in association with exposure to the different antibiotic compounds. The computer executable components further comprise a vectorization component that generates a target functional capacity vector for a target genome excluded from the reference data structure, and a susceptibility forecasting component that employs the reference data structure and the target functional capacity vector to determine one or more of the antibiotic compounds to which the target genome is susceptible. For example, in various implementations, the susceptibility forecasting component can employ one or more machine learning algorithms to facilitate determining the one or more antibiotic compounds based on degrees of similarity between the target functional capacity vector and the functional capacity vectors.

In some embodiments, elements described in connection with the disclosed systems can be embodied in different forms such as a computer-implemented method, a computer program product, or another form.

### DESCRIPTION OF THE DRAWINGS

FIG. 1 illustrates a high-level flow diagram of an example, non-limiting computer-implemented method for predicting biological reactions within a phenotypic space using dimensionally reduced coding vectors that represent the functions of relevant genes or proteins in the phenotypic space, in accordance with one or more embodiments.

FIG. 2 illustrates a block diagram of an example, non-limiting system that facilitates predicting biological reactions within a phenotypic space using dimensionally reduced coding vectors that represent the functions of relevant genes or proteins in the phenotypic space, in accordance with one or more embodiments.

FIG. 3 presents a table identifying example functional codes for some bacterial functions defined by a public genetic coding system, in accordance with one or more embodiments described herein.

FIG. 4 illustrates a flow diagram of an example, non-limiting computer-implemented method for representing a genome with a dimensionally reduced coding vector that represents one or more target functions associated with the genome within a target phenotypic space, in accordance with one or more embodiments.

FIG. 5 presents a table comprising example reference functional omics data for known bacterial genomes and a single antibiotic class, in accordance with one or more embodiments.

FIG. 6 illustrates a flow diagram of an example, non-limiting computer-implemented method for generating reference functional omics that facilitates predicting antibiotic resistance and complementary combinations of antibiotics, in accordance with one or more embodiments described herein.

FIG. 7 presents a table comprising example reference functional omics data for known genomes and two different antibiotic compounds, in accordance with one or more embodiments.

FIG. 8 illustrates a block diagram of an example, non-limiting system that facilitates predicting antibiotic resistance from functional omics data and recommending complementary combinations of antibiotics in accordance with one or more embodiments.

FIG. 9 illustrates a flow diagram of an example, non-limiting computer-implemented method for predicting antibiotic resistance from functional omics data and recommending complementary combinations of antibiotics in accordance with one or more embodiments.

FIG. 10 presents a table comprising functional omics data for an unknown genome and known genomes relative to a single antibiotic class, in accordance with one or more embodiments.

FIG. 11 illustrates an example matrix representing the distances between a functional capacity vector (FCV) for an unknown genome and the FCVs for known genomes, in accordance with one or more embodiments.

FIG. 12 demonstrates example clustering by FCVs for antibiotic resistance prediction, in accordance with one or more embodiments.

FIG. 13 illustrates a high-level flow diagram of an example, non-limiting computer-implemented method for identifying gene/protein sequences using dimensionally reduced coding vectors in accordance with one or more embodiments.

FIG. 14 illustrates a flow diagram of an example, non-limiting computer-implemented method for predicting antibiotic resistance from functional omics data in accordance with one or more embodiments.

FIG. 15 illustrates a flow diagram of another example, non-limiting computer-implemented method for predicting antibiotic resistance from functional omics data in accordance with one or more embodiments.

FIG. 16 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Technical Field or Summary sections, or in the Detailed Description section.

Any organism is a by-product of both its genetic makeup and its environment. For example, a specific type/species of bacteria can come in many different strain variations that exhibit different characteristics. For instance, one strain of Escherichia coli (E. coli) can cause an infected patient to experience no symptoms while another strain can produce ciguatoxin, causing an infected patient to experience severe clinical symptoms. These different characteristics are a result of both the organism's genetic makeup and its environment. In this regard, an organism's genotype refers to its genetic constitution, that is, its genes and the combination of alleles for each gene, which can vary between organisms of a same species. An organism's phenotype is the set of observable characteristics of the organism resulting from the interaction of its genotype with its environment. Biologists have historically considered an organism's gene or protein sequence as the driver of similarity, focusing on sequence variation as alleles. However, this approach neglects the true driver of phenotype which are the protein functional domains that have the capability of carrying out the enzymatic reactions.

This application relates to computer-implemented techniques for predicting biological reactions from functional omics data that captures the true driver of phenotypic variations amongst organisms. Omics aims at the collective characterization and quantification of pools of biological molecules that translate into the structure, function, and dynamics of an organism. For example, in contrast to genetics, which focuses on single genes, genomics focuses on all genes (genomes) and their inter-relationships. This approach allows studying how complex interactions between genes and molecules influence the phenotype. Functional genomics is a field of molecular biology that attempts to describe gene (and protein) functions and interactions.

Various embodiments of the disclosed subject matter combine functional genomics and machine learning techniques to facilitate predicting biological reactions related to antibiotic resistance and susceptibility and predicting complementary combinations of antibiotics. In one or more embodiments, the disclosed techniques use dimensionality reduction to vectorize an organism's genome, and more particularly selected proteins encoded in the organism's genome, using a functional capacity vector (FCV) that represents the functions of the selected proteins in a specific phenotypic space. For example, as applied to bacterial resistance prediction, in one implementation, the specific phenotypic space can encompass bacterial phenotypes associated with different phenotypic characteristics considered relevant to antibiotic resistance to one or more antibiotic compounds. In this regard, an FCV generated for a particular organism represents the roles that different proteins expressed by the organism play in association with creating is phenotypic characteristics relative to a particular environment (e.g., in-vivo, in-vivo and exposed to a particular antibiotic compound, etc.). As described in greater detail infra, the FCV captures the functions of the selected proteins based on the selected proteins comprising one or more functional domains that are responsible for one or more selected target functions or features considered relevant to the specific phenotypic space.

In various embodiments, the disclosed FCVs can be formed using existing functional omics data that identifies known functions or features of different protein domains and provides a standardized coding system that assigns distinct codes (e.g., numeric codes, alphanumeric codes, etc.) to the known functions or features. For example, the coding system can assign distinct codes to known phenotypic features, including (but not limited to), molecular function features, cellular component features, biological process features, and the like. One example of a suitable coding system that can be used to generate the FCVs includes the Gene Ontology™ coding system that assigns annotations referred to as GO terms (e.g., Gene Ontology terms) to different gene products (which include proteins, ribonucleic acid (RNA), etc.) and provides a statement about the function of the respective gene products. Other suitable coding systems can include the enzyme commission number (EC number) classification system and the InterPro™ classification system. In some embodiments, a single coding system can be used. In other embodiments, a plurality (two or more) coding systems can be used. It should be appreciated that the coding systems noted herein are merely exemplary and various other similar omics data coding systems can be used. In some embodiments, the specific coding system used can be selected based on the phenotypic space in question.

In this regard, in one or more embodiments, the process for generating an FCV for an organism's genome can include selecting a coding system for a set of possible phenotypes included within a target phenotypic space. One or more codes are then selected from the coding system based on the context of a particular phenotypic question or phenotype of interest. For example, in one or more implementations, the phenotypic question can generally encompasses determining whether and/or why the organism is resistant or susceptible to one or more antibiotic compounds. According to this example, the one or more codes that are selected can be based on the one or more codes representing functions or features that are considered relevant to the organism's resistance or susceptibility to the one or more antibiotic compounds. In some embodiments, the relevant functions or features can be determined using principal component analysis (PCA). The number of codes (respectively corresponding to the relevant functions or features) selected can vary. For example, in some implementations, one or a few codes may be selected. In other implementations, hundreds or thousands of codes may be selected.

The FCV generation process further includes, for each code, identifying one or more protein functional domains (also referred to generally herein as “functional domains”) annotated as being responsible for the feature/function represented by the code or otherwise associated with the feature/function represented by the code. In various implementations, the annotated functional domains can be identified using existing functional omics data that annotates protein domains with corresponding functions/features and/or the corresponding codes for the functions/features. All (or a filtered subset) of the proteins encoded by the genome that have the one or more functional domains are then identified and modeled as an FCV. This process can be performed for each selected code, resulting in a composite FCV for the genome that represents sets of proteins respectively having the functional domains responsible for (or otherwise attributed to) the corresponding functions/feature of the selected codes. In this regard, the FCV generation process vectorizes an organism's genome, and more particularly selected proteins encoded in the genome, replacing using the functional capacity vector (FCV) as a new representation of the selected proteins (instead of the gene or protein sequence). This is a form of dimensionality reduction in the relevant coding space.

In various embodiments, the disclosed techniques can be applied to facilitate predicting antibiotic resistance by generating FCVs for different bacterial genomes whose antimicrobial resistance (AMR) status against one or more antibiotic compounds is known. For example, the AMR status for each (or in some implementations one or more) of the different known bacterial genomes can indicate what antibiotic compounds each genome is resistant or susceptible to, and in some implementations, the minimum inhibitory concentrations (MICs) for the antibiotic compounds (e.g., relative to an infected human or another host). The FCVs for the different bacterial genomes can in this context, represent the functions that relevant encoded proteins play in causing their respective phenotypes (e.g., their in-vivo behavior, their different AMR statuses when exposed to a same antibiotic compound, etc.). In this regard, the FCVs for the different bacterial genomes in this context correlate antibiotic resistance to specific protein domains. The collective information for the known genomes (generally referred to herein as the reference data), including their FCVs and AMR statuses can then be used to facilitate predicting antibiotic resistance and susceptibility for new bacterial genomes whose AMR status is unknown. For example, in some embodiments, the reference data can be used to predict antibiotic compounds that the new bacterial genome will be susceptible to and/or resistant to based on similarities between an FCV generated for the new bacterial genome and the FCVs of the known genomes. In addition, in implementations in which the AMR status provides the MICs for the antibiotic compounds, the reference data can also be used to predict the MIC for one or more antibiotic compounds against the new bacterial genome.

The reference data developed for the known genomes can also be used to predict complementary combinations of antibiotic compounds (e.g., that are likely to be more effective together than alone for treating certain bacterial infections) based on variations between FCVs and AMR statuses for different genome when exposed to different antibiotic compounds. For example, in one or more embodiments, if for two different antibiotic compounds, the change in FCVs is in opposite directions for resistant and susceptible genomes, then those two antibiotic compounds can be expected to work better in combination.

Various embodiments of the disclosed techniques for generating and applying FCVs are described with reference to bacterial genomes and predicting antibiotic resistance and complementary antibiotics. However, the disclosed subject matter is not limited this domain. In this regard, the disclosed techniques can be used to generate FCVs for various species for identifying correlations between the functions of relevant genes and proteins in a target phenotypic space.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details. It is noted that the drawings of the present application are provided for illustrative purposes only and, as such, the drawings are not drawn to scale.

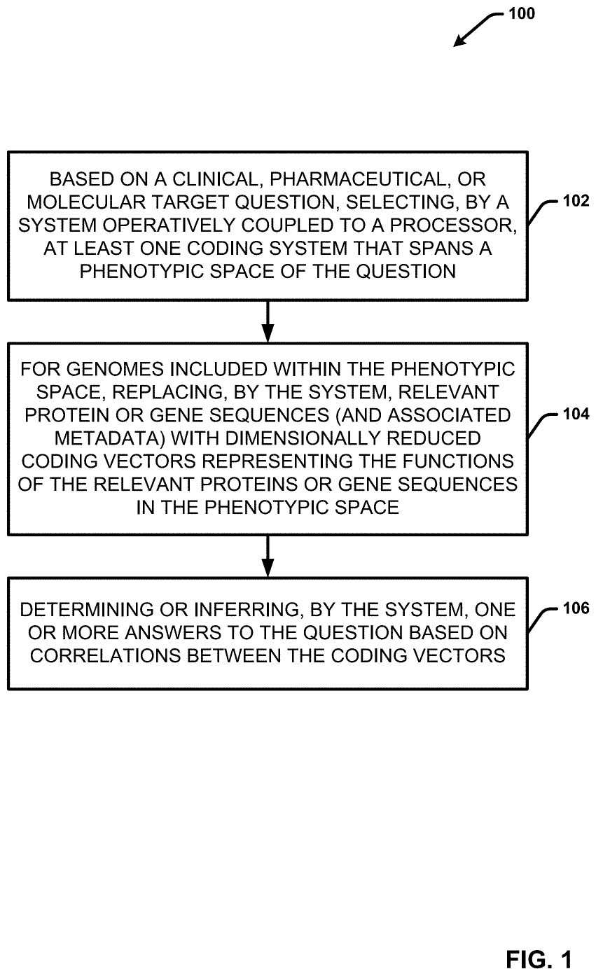

FIG. 1 illustrates a high-level flow diagram of an example, non-limiting computer-implemented method 100 for predicting biological reactions within a phenotypic space using dimensionally reduced coding vectors (e.g., FCVs) that represent the functions of relevant genes or proteins in the phenotypic space, in accordance with one or more embodiments. Method 100 provides a high-level overview of the disclosed computer-implemented techniques for predicting biological reactions from functional omics data that captures the true driver of phenotypic variations amongst organisms.

In accordance with method 100, at 102, based on a clinical, pharmaceutical, or molecular target question, a system operatively coupled to a processor can select at least one coding system that spans a phenotypic space of the question. At 104, for genomes included within the phenotypic space, the system can replace relevant protein or gene sequences (and associated metadata) with dimensionally reduced coding vectors (referred to herein as functional capacity vectors or FCVs) representing the functions of the relevant proteins or gene sequences in the phenotypic space. At 106, the system can then determine or infer (e.g., using one or more machine learning tools), one or more answers to the question based on correlations between the coding vectors.

Method 100 can be applied to various types of genomes for identifying correlations between the functions of relevant genes and proteins in a phenotypic space of interest. For example, in various embodiments, method 100 can be applied to predict antibiotic resistance and predict complementary antibiotic complementary antibiotic combinations optimize treatment of bacterial infections. For instance, in one example use case, method 100 can be applied to evaluate a patient's bacterial infection to determine or infer what antibiotics the infection is resistant to and what antibiotics the bacterial infection is susceptible to. In this regard, antibiotics have historically been prescribed based on the genome of the organism responsible for the infection as represented by the name of the organism (e.g., E. coli, streptococcus, staphylococcus, Pseudomonas aeruginosa, enterococci, etc.). However, the name of the organism does not provide an indication of its' phenotype or provide an adequate indication as to whether the organism is going to susceptible or resistant to a particular antibiotic. As described in greater detail infra, in various embodiments, the techniques outlined by method 100 can be applied to predict antibiotic compounds that a new bacterial genome (e.g., of the organism infecting a patient) will be susceptible to and/or resistant to base on similarities between an FCV generated for the new bacterial genome and the FCVs of bacterial genomes whose AMR status relative to various antibiotics is known. The disclosed techniques thus providing a significantly more targeted approach for determining the best antibiotic treatment and discouraging the evolution of bacterial resistance. In addition, based on variations between FCVs for bacterial genomes and their AMR status for different antibiotic compounds, the techniques outlined by method 100 can also be used to design new antibiotics (e.g., designed to target the relevant functional domains) and predict complementary combinations of antibiotic compounds that will have increased efficacy and discourage the evolution of bacterial resistance. Thus, in accordance with some example implementation, the techniques outlined by method 100 can be applied to provide substantial clinical improvements in optimizing clinical treatment and minimizing the development of multidrug-resistant (MDR), bacteria which has become a serious global threat.

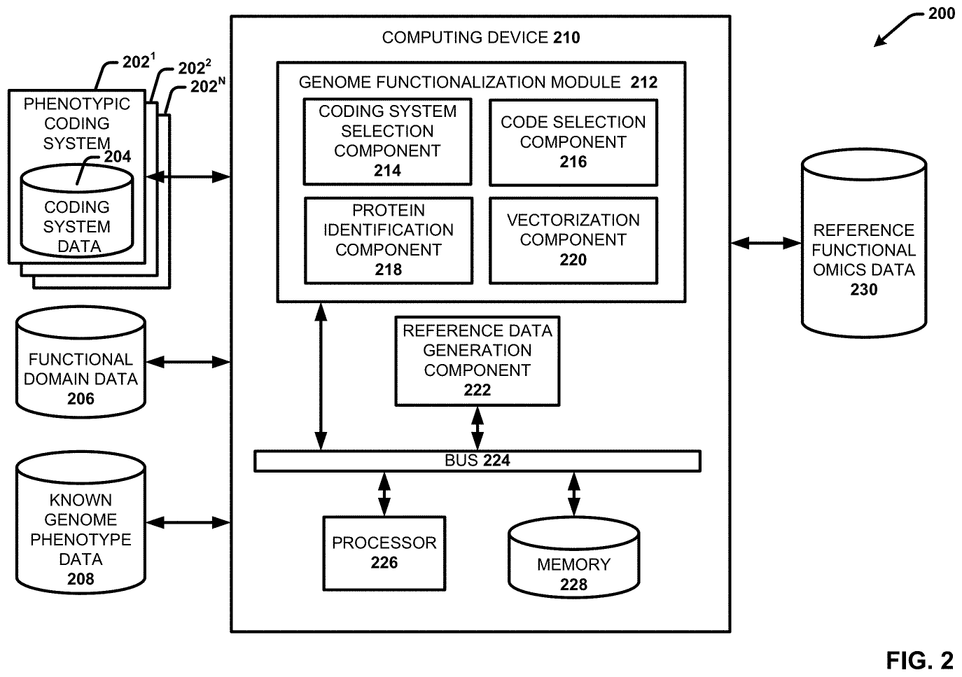

FIG. 2 illustrates a block diagram of an example, non-limiting system 200 that facilitates predicting biological reactions within a phenotypic space using dimensionally reduced coding vectors that represent the functions of relevant genes or proteins in the phenotypic space, in accordance with one or more embodiments. For example, in some embodiments, system 200 can perform one or more functions of method 100, as well as additional functions described herein.

Embodiments of systems described herein can include one or more machine-executable components embodied within one or more machines (e.g., embodied in one or more computer readable storage mediums associated with one or more machines). Such components, when executed by the one or more machines (e.g., processors, computers, computing devices, virtual machines, etc.) can cause the one or more machines to perform the operations described. For example, in the embodiment shown, system 200 includes a computing device 210 that includes a genome functionalization module 212 and a reference data generation component 222. The genome functionalization module 212 further includes a coding system selection component 214, a code selection component 216, a protein identification component 218 and a vectorization component 220. In this regard, the genome functionalization module 212 itself, the components associated therewith (e.g., the coding system selection component 214, the code selection component 216, the protein identification component 218 and the vectorization component 220), and the reference data generation component 222 can respectively be or correspond to machine or computer executable components.

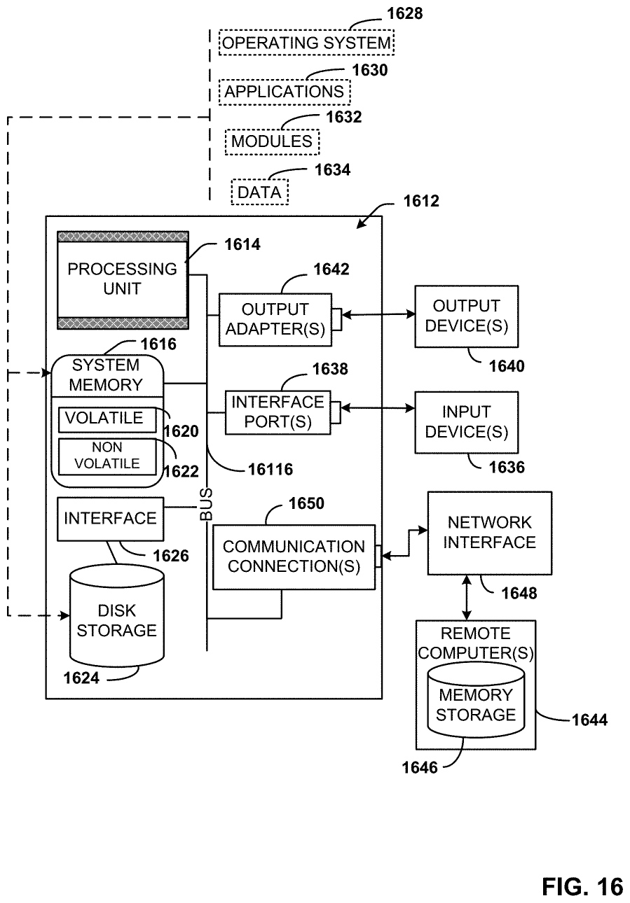

The computing device 210 can further include or be operatively coupled to at least one memory 228 and at least one processor 226. In various embodiments, the at least one memory 228 can store executable instructions (e.g., embodied by the genome functionalization module 212 itself, the respective components associated therewith, the reference data generation component 222, and additional components described herein) that when executed by the at least one processor 226, facilitate performance of operations defined by the executable instructions. In some embodiments, the memory 228 can also store the various data sources and/or structures of system 200 (e.g., data sources including but not limited to, coding system data 204, functional domain data 206, known genome phenotype data 208, reference functional omics data 230, and the like). In other embodiments, the various data sources and structures of system 200 can be stored in other memory one or more remote device or systems that are accessible to the computing device 102 (e.g., via one or more networks). The computing device 210 can further include a device bus 224 that communicatively couples the various components of the computing device 210. Examples of said processor 226 and memory 228, as well as other suitable computer or computing-based elements, can be found with reference to FIG. 16 with respect to processing unit 614 and system memory 116, and can be used in connection with implementing one or more of the systems or components shown and described in connection with FIG. 1 or other figures disclosed herein.

System 200 also includes various electronic data sources and/or data structures comprising information that can be read by, used by and/or generated by the genome functionalization module 212 and the reference data generation component 222. For example, as shown in system 200, these data sources and/or data structures can include but are not limited to: one or more phenotypic coding systems 202 respectively providing a database (or another suitable data source or structure) comprising coding system data 204, a database (or another suitable data source or structure) providing functional domain data 206, a database (or another suitable data source or structure) providing known genome phenotype data 208 (e.g., AMR status and optionally MIC values for known bacterial genomes against one or more antibiotic compounds), and database (or another suitable data source or structure) providing reference functional omics data 230.

In some embodiments, computing device 210 can comprise any type of component, machine, device, facility, apparatus, and/or instrument that comprises a processor and/or can be capable of effective and/or operative communication with a wired and/or wireless network. All such embodiments are envisioned. For example, the computing device 210 can comprise a server device, a computing device, a general-purpose computer, a special-purpose computer, a tablet computing device, a handheld device, a server class computing machine and/or database, a laptop computer, a notebook computer, a desktop computer, a cellular phone, a smart phone, a consumer appliance and/or instrumentation, an industrial and/or commercial device, a digital assistant, a multimedia Internet enabled phone, a multimedia player, and/or another type of device.

It should be appreciated that the embodiments of the subject disclosure depicted in various figures disclosed herein are for illustration only, and as such, the architecture of such embodiments are not limited to the systems, devices, and/or components depicted therein. For example, although system 200 depicts a single computing device 210 for execution of the various computer executable components (e.g., the genome functionalization module 212 itself, the respective components associated therewith, and the reference data generation component 222, and additional components described herein), in some embodiments, one or more of the components can be executed by different computing devices (e.g., including virtual machines) separately or in parallel in accordance with a distributed computing system architecture. In additions, in some embodiments, system 200 can comprise various additional computer and/or computing-based elements described herein with reference to operating environment 1600 and FIG. 16. In several embodiments, such computer and/or computing-based elements can be used in connection with implementing one or more of the systems, devices, components, and/or computer-implemented operations shown and described in connection with FIG. 1 or other figures disclosed herein.

In some embodiments, the computing device 210 can be coupled (e.g., communicatively, electrically, operatively, etc.) to one or more external systems, data sources, and/or devices (e.g., the one or more phenotypic coding systems 202 and the associated coding system data 204, the functional domain data 206, the known genome phenotype data 208, the reference functional omics data 230, other computing devices, communication devices, etc.) via a data cable (e.g., coaxial cable, High-Definition Multimedia Interface (HDMI), recommended standard (RS) 232, Ethernet cable, etc.). In some embodiments, the computing device 210 can be coupled (e.g., communicatively, electrically, operatively, etc.) to one or more external systems, sources, and/or devices (e.g., the one or more phenotypic coding systems 202 and the associated coding system data 204, the functional domain data 206, the known genome phenotype data 208, the reference functional omics data 230, other computing devices, communication devices, etc.) via a network.

According to multiple embodiments, such a network can comprise wired and wireless networks, including, but not limited to, a cellular network, a wide area network (WAN) (e.g., the Internet) or a local area network (LAN). For example, the computing device 210 can communicate with one or more external systems, sources, and/or devices, for instance, computing devices (and vice versa) using virtually any desired wired or wireless technology, including but not limited to: wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Zigbee and other 802.XX wireless technologies and/or legacy telecommunication technologies, BLUETOOTH®, Session Initiation Protocol (SIP), ZIGBEE®, RF4CE protocol, WirelessHART protocol, 6LoWPAN (IPv6 over Low power Wireless Area Networks), Z-Wave, an ANT, an ultra-wideband (UWB) standard protocol, and/or other proprietary and non-proprietary communication protocols. In such an example, the computing device 210 can thus include hardware (e.g., a central processing unit (CPU), a transceiver, a decoder), software (e.g., a set of threads, a set of processes, software in execution) or a combination of hardware and software that facilitates communicating information between the computing device 210 and external systems, sources, and/or devices (e.g., the one or more phenotypic coding systems 202 and the associated coding system data 204, the functional domain data 206, the known genome phenotype data 208, the reference functional omics data 230, other computing devices, communication devices, etc.).

The genome functionalization module 212 can provide for generating one or more functional capacity vectors (FCVs) to represent an organism's genome. In various embodiments, the genome functionalization module 212 can generate the disclosed FCVs using existing functional omics data that identifies known functions or features of different genes and/or proteins and provides a standardized coding system that assigns distinct codes (e.g., numeric codes, alphanumeric codes, etc.) to the known functions or features. In the embodiment shown in FIG. 2, this functional omics data can be provided by one or more phenotypic coding systems 202^{1-N} (e.g., a coding system data 204).

For example, various omics data coding systems have been developed that provide a defined coding scheme for identifying known functions of genes for various species. On example of such a coding system includes the Gene Ontology Resource coding system, which provides an open source knowledge base of information on the functions of genes. The Gene Ontology Resource™ system assigns distinct codes, referred to as GO terms, to different gene products (which include proteins, ribonucleic acid (RNA), etc.) and provides annotations for the GO terms that include statements about the function of the respective gene products. These annotations include molecular function annotations that describe the molecular function of individual gene products, cellular component annotations that describe where the gene products are active, and biological process annotations that describe the pathways and larger processes to which that gene product's activity contribute.

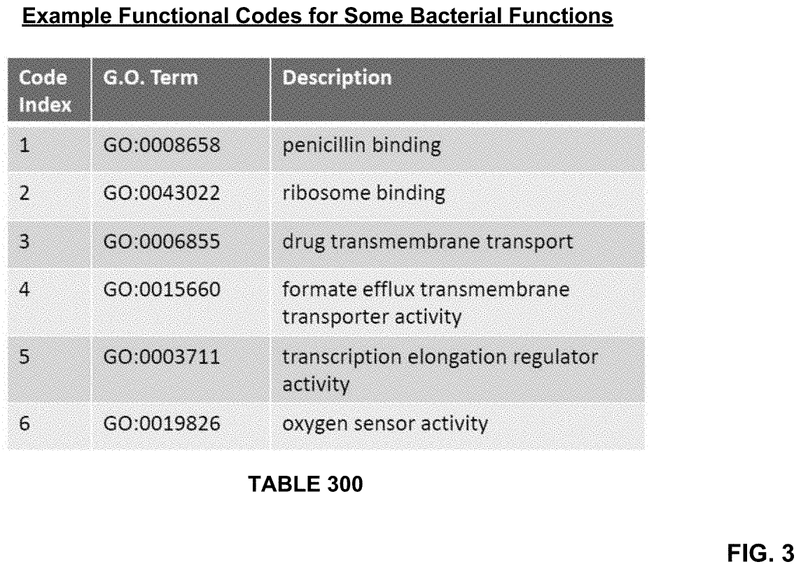

For example, FIG. 3 presents a table 300 identifying some example functional codes (e.g., GO terms) for some bacterial features/functions defined by the Gene Ontology Resource™ coding system, in accordance with one or more embodiments described herein. As shown in table 300, the GO terms consist of unique numeric identifiers that describe a specific molecular function, cellular component, or biological processes provided by one or more gene products (e.g., proteins) encoded by one or more genes of a known genome. In this regard, each GO term represents a known molecular “feature” of a particular gene with respect to a particular species genome. In the embodiment shown, only six GO terms associated with bacterium genomes are provided for exemplary purposes. These GO terms are respectively identified and referred to herein as codes 1-6, respectively. However, in practice, the number of GO terms or functional codes associated with a particular genome can include hundreds or thousands (or more). For example, as of February of 2020, the Gene Ontology Resource™ coding system included 44,579 GO terms and 7,400,326 annotations, for 1,359,256 gene products and 4,591 species.

With reference again to FIG. 2, in various embodiments, the one or more phenotypic coding systems 202^{1-N} can include the Gene Ontology Resource™ coding system. With these embodiments, the coding system data 204 can include the GO terms and annotations for the numerous gene products and species. However, Gene Ontology Resource™ coding system merely provide one example of a suitable coding system that can be employed by genome functionalization module 212 to generate the FCVs. Other suitable coding systems can include the enzyme commission number (EC number) classification system, the InterPro™ classification system, and similar coding systems. The number of different phenotypic coding systems 202^{1-N} can vary.

In some embodiments, the specific phenotypic coding system to be used by the genome functionalization module 212 can be predefined. With these embodiments, the genome functionalization module 212 can receive or otherwise access the specific phenotypic coding system that has been predefined for creating FCVs for one or more genomes, wherein the predefined phenotypic coding system includes codes for defined phenotypic characteristics (e.g., molecular functions or features) that are associated with a target phenotypic space or target set of possible phenotypes for the one or more genomes. For example, as applied to bacterial resistance prediction, in one implementation, the target phenotypic space can encompass bacterial phenotypes associated with different phenotypic characteristics considered relevant to antibiotic resistance and/or susceptibility to a specific antibiotic compound or specific class of antibiotic compounds, or a variety of different antibiotic compounds in general.

In other embodiments, the genome functionalization module 212 can be configured to select one or more appropriate phenotypic coding systems from amongst the phenotypic coding systems 202^{1-N} that provides codes for the appropriate biological functions or features that that are relevant to particular genome and/or target phenotypic space to be represented by the FCV. With these embodiments, the genome functionalization module 212 can include coding system selection component 214 to select or facilitate selecting one or more appropriate coding system from amongst the phenotypic coding systems 202^{1-N} based on a target phenotypic space being evaluated. For example, the coding system selection component 214 can receive information identifying or otherwise indicating the target phenotypic space and select one or more of the phenotypic coding systems 202^{1-N} that includes information identifying gene and/or gene product functions or features that are relevant to the target phenotypic space. In some embodiments, a single coding system can be selected. In other embodiments, a plurality (two or more) coding systems can be selected.

For instance, as applied to bacterial resistance prediction, in one implementation, the target phenotypic space can encompass bacterial phenotypes associated with different phenotypic characteristics considered relevant to antibiotic resistance and/or susceptibility to a specific antibiotic compound, a specific class of antibiotic compounds, or a variety of different antibiotic compounds in general. In another example, implementation as applied to bacterial resistance prediction, the target phenotypic space can more specifically identify a subset of bacterial phenotypes that vary with respect to a particular type of biological characteristic. For example, different bacterial phenotypes can vary based on a variety of characteristics, including molecular function characteristics, cellular function characteristics, biological process characteristics and the like. Thus, in one example, implementation, target phenotypic space can be restricted to one of these types of characteristics. For example, if bacterial resistance to a particular antibiotic compound or group of antibiotic compounds has been determined to be attributed to an enzymatic issue, then the target phenotypic space can be refined to encompass phenotypes that vary with respect to different biological process characteristics. With this example, the coding system selection component 214 can select an appropriate coding system that provides information identifying different biological process characteristics associated with bacterial genome products (e.g., proteins) and assigns distinct codes to the different biological process characteristics. In another example, if bacterial resistance to a particular antibiotic compound or group of antibiotic compounds has been determined to be attributed to an organelle issue, then the target phenotypic space can be refined to encompass phenotypes that vary with respect to different cellular component characteristics. With this example, the coding system selection component 214 can select an appropriate coding system that provides information identifying different cellular component characteristics associated with bacterial genome products (e.g., proteins) and assigns distinct codes to the different biological process characteristics.

In some embodiments, the coding system selection component 214 can be configured to apply predefined requirements or restrictions in association with selecting the one or more appropriate phenotypic coding systems from amongst the phenotypic coding systems 202^{1-N}. For example, as omics research evolves, existing phenotypic coding systems will continue to grow and adapt their taxonomies and new coding systems may emerge which can vary in structure, architecture and the like. In this regard, in some implementations, the predefined requirements or restrictions can specify a type of structure or architecture required for the coding system taxonomy. For example, in one implementation, system 200 can require the taxonomy to be structured as an acyclic graph. Thus, in some embodiments, the coding system selection component 214 can select the appropriate phenotypic coding system from amongst the phenotypic coding systems 202^{1-N} based on the phenotypic coding system meeting the one or more predefined requirements (e.g., based on the coding system having a specific taxonomy structure or the like).

Whether selected or assigned (e.g., predefined), the phenotypic coding system 202 provides the coding system data 204 that is used by the genome functionalization module 212 to generate FCVs for one or more genomes, that is, a set of codes respectively corresponding to known gene and/or gene product (e.g., proteins, RNA, etc.) functions or features associated with a target phenotypic space. The genome functionalization module 212 further includes code selection component 216, protein identification component 218 and vectorization component 220 to facilitate the FCV generation process using one or more of the selected or assigned phenotypic coding systems 202^{1-N}.

In this regard, at a high level, in various embodiments, in order to generate an FCV to represent an organism's genome function relative to a target phenotypic space, the code selection component 216 can receive or select one or more codes from the one or more phenotypic coding systems 202^{1-N} that are considered relevant to the target phenotypic space or a phenotype of interest. For each selected code (or in some implementations one or more of the selected codes), the protein identification component 218 can identify one or more protein functional domains associated with the code as identified in existing functional omics data (e.g., the functional domain data 206 and/or the coding system data 204). For example, in one or more embodiments, the functional domain data 206 (and/or the coding system data 204) can include information identifying known protein functional domains and specific features and/or functions (e.g., molecular functions, cellular components, biological processes, etc.) that the respective functional domains are responsible for or otherwise associated with. With these embodiments, the protein identification component 218 can identify the one or more protein functional domains associated with the code based information provided in the functional domain data 206 (and/or the coding system data 204) that annotates the one or more protein functional domains with the code and/or the feature/function represented by the code

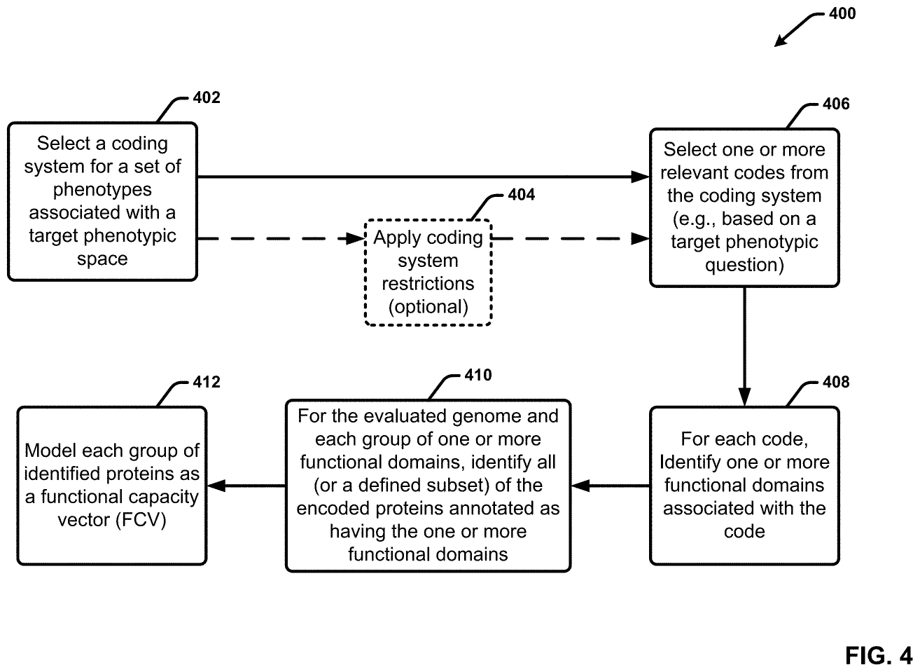

After the one or more functional domains have been identified, for the particular genome being processed (e.g., the genome for which an FCV is being generated), the protein identification component 218 can further identify one or more proteins encoded by the genome annotated that have the one or more functional domains. The vectorization component 220 can further model these one or more proteins as an FCV. This process can be performed for each selected code, resulting in a composite FCV for the genome that represents sets of proteins respectively having the functional domains responsible for (or otherwise attributed to) the corresponding functions/feature of the selected codes. Additional features and functionalities the code selection component 216, the protein identification component 218 and the vectorization component 220 are described in greater detail with reference to FIG. 4.

FIG. 4 illustrates a flow diagram of an example, non-limiting computer-implemented process 400 for representing a genome with an FCV, in accordance with one or more embodiments. In this regard, process 400 provides a high-level flow diagram of an example process that can be performed by the genome functionalization module 212 to generate an FCV for any genome that reflects the relevant functions of that genome in a target phenotypic space.

With reference to FIG. 4 in view of FIG. 2, in one or more embodiments, the process for generating an FCV for an organism's genome can begin at 402 with selecting a coding system (e.g., one or more of the phenotypic coding systems 102^{1-N}) for a set of phenotypes associated with a target phenotypic space. In accordance with these embodiments, the coding system selection component 214 can perform the task of coding system selection by selecting one or more suitable phenotypic coding system from amongst the one or more phenotypic coding systems 202^{1-N} as described with reference to FIG. 2. For example, as applied to bacterial resistance prediction, in one implementation, the target phenotypic space can encompass bacterial phenotypes associated with different phenotypic characteristics considered relevant to antibiotic resistance to one or more antibiotic compounds. According to this example, the appropriate coding system should identify and assign distinct codes to various features or functions of gene and/or the gene products (e.g., proteins) that have been identified as being encoded in one or more bacterial genomes and that may influence antibiotic resistance to the one or more antibiotic components. In some implementations in which coding system restrictions are defined, at 404, the coding system selection component 214 can apply the coding system restrictions in association with selecting the coding system. In other embodiments, the phenotypic coding system to be used by the genome functionalization module 212 for FVC can be preselected. With these embodiments, the coding system selection steps can be skipped.

At 406, the code selection component 216 can select one or more relevant codes from the coding system based on a target phenotypic question. In particular, the code selection component 216 can select one or more codes that reflect one or more target characteristics (e.g., functions or features) included in the coding system data 204 that are considered most relevant or most important to the function of the genome with respect to the target phenotypic space (or the context of a particular phenotypic question). For example, with respect to bacterial resistance of a bacterial genome to various types of antibiotic compounds in general, the code selection component 216 can be configured to select one or more codes that represent features or functions considered most relevant to bacterial resistance or susceptibility in general. In another example, with respect to bacterial resistance of a bacterial genome to a specific type of antibiotic compound, the code selection component 216 can be configured to select one or more codes that represent features or functions considered most relevant to bacterial resistance or susceptibility to the specific type of antibiotic compound.

In this regard, the coding system codes represent feature or functions of proteins (and in some implementations other gene products, including RNA and the like) encoded by one or more genomes included in the target phenotypic space. For example, with reference again to FIG. 3, code 1 (corresponding to GO term GO:0008658) represents the function of penicillin binding; Code 2 (corresponding to GO term GO:0043033) represents the function of ribosome binding; Code 3 (corresponding to GO term GO:0006855) represents the drug/medication transmembrane transport; Code 4 (corresponding to GO term GO:0015660) represents the function of formate efflux transmembrane transport activity; Code 5 (corresponding to GO term GO:0003711) represents the function of transcription elongation regulator activity; and Code 6 (corresponding to GO term GOL0019826) represents the function of oxygen sensor activity. The goal of code selection at 406 is to select a subset of codes from the set of codes provided by the coding system that represent the functions or features that are considered most relevant to the target phenotypic question or a target phenotype. For example, with respect to bacterial resistance analysis, the target phenotypic question can include: “What phenotypic features/functions are most relevant to antimicrobial resistance or susceptibility of any bacterial organism to any antibiotic compound?”, or “What features/functions are most relevant to antimicrobial resistance or susceptibility of gram negative bacteria to antibiotic resistance or susceptibility to beta-lactam (B-lactam) antibiotics?”. In this regard, the code selection at 406 corresponds to feature selection.

The techniques employed by the code selection component 216 to determine which codes to select can vary. In some embodiments, the relevant features/functions to a particular phenotypic question or target phenotype can be predefined. According to these embodiments, the code selection component 216 can be configured to select those codes from the coding system that represent previously identified relevant features/functions to the target phenotypic question. For example, in one or more implementations, the code selection component 216 can receive or otherwise access feature information that identifies important molecular functions or features that have been previously correlated to the target phenotypic question (e.g., functions or features relevant to bacterial resistance and/or susceptibility of one or more types of bacteria to one or more antibiotic compounds). According to this example, the code selection component 216 can receive the feature information identifying the relevant features or functions and then select the corresponding codes for those relevant features/functions in the coding system.

In some implementations of these embodiments, the relevant features/functions can be determined using PCA analysis (e.g., performed by the code selection component 216 or another component or system). In some implementations, in which PCA analysis is used, the coding system selection component 214 can also employ a defined thresholding scheme to select only those features/functions that have coefficients above a defined threshold. Alternatively, the coding system selection component 214 can be configured to select only the top N features/functions (e.g., the top 50, 100, etc.). Still in other embodiments, the code selection component 216 can employ various additional machine learning techniques to identify the most relevant features/functions to a particular phenotypic space or question being evaluated using evidence based biological reaction data provided in various electronic data sources and system accessible to the computing device 210 (e.g., white papers, literature, articles, and other scientific research documents).

At 408, for each selected code, the protein identification component 218 can then identify one or more functional domains associated with code (e.g., for each of the one or more codes selected at 406). For example, a single protein can have more than one functional domain, (although some proteins can have a single functional domain), and each (or in some implementations one or more) functional domain can be responsible for a particular function or feature (or otherwise be associated with a particular function/feature). In this regard, a single protein can include different functional domains that respectively provide different functions/features, wherein at least some of the functions or features have been identified in the coding system data. In this regard, the protein identification component 218 can employ existing functional omics data that identifies known functions/features associated with different protein domains. For example, in the embodiment shown in FIG. 2, this information is represented by functional domain data 206. In another embodiments, this information can be included with the coding system data 204. Regardless of the source of the functional domain data 206, for each selected code, the protein identification component 218 can employ the functional domain data 206 to identify one or more functional domains that are annotated with information that identifies or indicates that the respective functional domains provide (or are otherwise associated with) the feature/function represented by the code.

At 410, for the evaluated genome and each group of one or more functional domains (e.g., associated with a single code), the protein identification component 218 can further identify all (or a defined subset) of the proteins encoded by that genome that are annotated as having the one or more functional domain. For example, the protein identification component 218 can determine or receive information identifying proteins encoded by the genome and/or the functional domains of the respective proteins. For example, in some implementations, the protein identification component 218 can determine or receive information for a genome that identifies all proteins encoded by the genome. The functional domain data 206 can also include information identifying known proteins and known functional domains for those known proteins. With this example implementation, the protein identification component can thus examine all (or a defined subset) of the proteins encoded by the genome and using the functional domain data 206, identify any (or a defined subset) of those proteins that have the specific functional group (or groups) corresponding to the function/feature represented by a selected code.

At 412, the vectorization component 220 can further model the group of identified proteins as an FCV. For example, in various embodiments, the vectorization component 220 can create an FCV for the genome that reflects the number of proteins identified as including the one or more functional domains. In another embodiment, the vectorization component 220 can create an FCV for the genome that reflects the frequency with which the one or more functional domains appear in the genome.

In some implementations, at 410 the protein identification component 218 may determine that the genome does not encode any proteins which include the one or more functional domains associated with a particular code. With these implementations, the resulting FCV can indicate that the genome lacks the particular function or feature.

The protein identification component 218 and the vectorization component 220 can respectively repeat the processes performed from 408 to 412 for each selected code, resulting in a composite FCV for the genome that represents sets of proteins respectively having the functional domains responsible for (or otherwise attributed to) the corresponding function/feature of the selected codes. In this regard, the FCV generation process vectorizes an organism's genome, and more particularly selected proteins encoded in the genome, replacing using the functional capacity vector (FCV) as a new representation of the selected proteins (instead of the gene or protein sequence) that represents the functions of the selected proteins in a specific phenotypic space. This is a form of dimensionality reduction in the relevant coding space.

With reference again to FIG. 2, in various embodiments, FCVs generated in accordance with process 400 can be used to generate inferences (e.g., for clinical, pharmaceutical and other molecular target questions) based on identified correlations between FCVs for different genomes relative to a particular phenotypic space. With these embodiments, the computing device 210 can include reference data generation component 222 to generate training or reference data for a distribution of genomes associated with a particular phenotypic space for which the answer to the target biological, clinical or pharmaceutical questions is known (e.g., provided in the known genome phenotype data 208). For example, the training or reference data can identify a known set of genomes, their respective FCVs that reflect their functional capacity in a particular phenotypic space in question (e.g., determined by the reference data generation component 222 using the genome functionalization module 212 and process 400), and the known answer to the target phenotypic question. In the embodiment shown, this training or reference data generated by the reference data generation component 222 is referred to as reference functional omics data 230.

For example, in some embodiments, the disclosed techniques can be applied to facilitate predicting antibiotic resistance by generating FCVs for different bacterial genomes whose antimicrobial resistance (AMR) status against one or more antibiotic compounds is known. With the embodiments, the known genome phenotype data 208 can include information identifying known (e.g., public) bacterial genomes and their known AMR status. For example, the known AMR status information for each (or in some implementations one or more) of the different known bacterial genomes can indicate what antibiotic compounds each of the genomes are resistant or susceptible to.

It should be appreciated that bacterial resistance and susceptibility are relative terms that are based the organism's environment (e.g., in vitro, in-vivo, the specific infected subject, etc.), the concentration of antibiotic compound applied, and the frequency of application. For example, a bacterial organism in an infected subject can demonstrate varying levels of resistance or susceptibility to different antibiotic compounds, which can be dependent on the concentration of the antibiotic compound applied, the frequency of application, and infected subject's species, age, size, level of infection, and the like. Antibiotic resistance and susceptibility can be measured in various ways. One standard metric used to evaluate antibiotic resistance and susceptibility is the minimum inhibitory concentration (MIC), which represents the lowest antibiotic concentration that prevents visible growth of the organism. Another metric includes the minimum bactericidal concentration (MBC), which is the lowest concentration of an antibacterial agent required to kill a particular bacterium.

As used herein, the term “resistant” with respect to bacterial resistance relative to an antibiotic compound indicates that the bacterial organism exhibits a level of resistance that exceeds a minimum level of resistance using a defined AMR metric under a defined context (e.g., in-vitro, in-vivo, in an adult human, etc.). The defined metric and context can vary. For example, in one or more implementations, a bacterial genome can be classified as resistant to an antibiotic compound if the MIC for the antibiotic compound when administered to the organism in a defined context (e.g., with respect to the infected subject and frequency of administration) exceeds a defined maximum concentration (e.g., a concentration considered unhealthy or toxic), or when any amount of the antibiotic component is ineffective at inhibiting growth of the organism. Likewise, as used herein, the term “susceptible” with respect to bacterial susceptibility relative to an antibiotic compound indicates that the bacterial organism exhibits a level of susceptibility that exceeds a minimum level of susceptibility using a defined AMR metric under a defined context (e.g., in-vitro, in-vivo, in an adult human, etc.). The defined metric and context can vary. For example, in one or more implementations, a bacterial genome can be classified as susceptible to an antibiotic compound if the MIC for the antibiotic compound when administered to the organism in a defined context is less a defined maximum concentration.

In some implementations the AMR status information can further identify the MICs for the different antibiotic compounds determined for the different genomes relative to a defined testing environment (e.g., for an infected human and/or or another defined host). In this regard, it should be appreciated that the MIC value for a particular antibiotic compound can vary based on the species and size of the infected subject (e.g., mammalian or other).

In accordance with these embodiments, the reference data generation component 222 can employ the genome functionalization module 212 to generate FCVs for the known bacterial genomes. For example, the FCVs for the known bacterial genomes can in this context, represent the functions that relevant encoded proteins play in causing their respective phenotypes (e.g., their in-vivo behavior, their different AMR statuses when exposed to a same antibiotic compound, etc.). In this regard, the FCVs for the different bacterial genomes in this context correlate antibiotic resistance to specific protein domains. The reference data generation component 222 can further generate reference functional omics data 230 for the purpose of generating inferences regarding bacterial resistance and/or complementary antibiotics using the collective information for the known genomes (generally referred to herein as the reference data). For example, in accordance with these embodiments, the reference functional omics data 230 would include information identifying known bacterial genomes, their AMR statuses for one or more antibiotic compounds, and their FVCs (e.g., relative to each of the antibiotic compounds). This reference functional omics data 230 can then be used to facilitate predicting antibiotic resistance and susceptibility for new bacterial genomes whose AMR status is unknown.

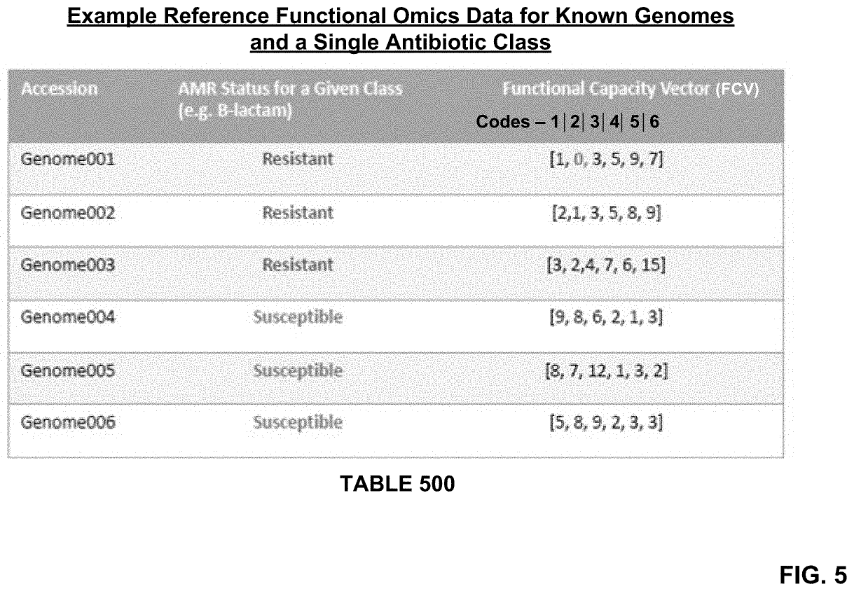

For example, FIG. 5 presents a table 500 comprising example reference functional omics data for known bacterial genomes and a single antibiotic class (B-lactam), in accordance with one or more embodiments. The reference functional omics data provided in table 500 demonstrates example reference functional omics data that can be generated by the reference data generation component 222 that can be used to predict antibiotic resistance of unknown genomes and/or to predict complementary antibiotics. In the embodiments shown, the reference functional omics data includes information identifying six known bacterial genomes, respectively identified as Genomes 001-006 and their respective AMR status relative to a particular class of antibiotics known as B-lactam. In this example, the AMR status indicates whether the respective genomes are either resistant or susceptible to the antibiotic. In some implementations, the AMR status can include an MIC value (or another metric) that reflects the degree of resistance or susceptibility of the genome to the antibiotic component (e.g., in a defined testing context). In this regard, the higher the MIC value, the greater level of antibiotic resistance.

The reference functional omics data in Table 500 further incudes the FCVs determined for each genome (e.g., by the reference data generation component 222 using the genome functionalization module 212). In accordance with this example, the FCVs are based on the six example functional codes shown in Table 300 (FIG. 3), respectively identified as codes 1-6. In this regard, the example FCVs are a length of six (because six codes were used to create the FCVs). It should be appreciated however that the number of codes/features evaluated can vary, and thus the length of the FCVs can also vary. For example, in some implementations, the number of codes/features evaluated can include hundreds or thousands of codes. In accordance with this example implementation, the FCVs reflect the frequency with which the corresponding functional domain for each code appears in the genome. For example, the FCV for Genome 001 is [1,0,3,5,9,7], which means that Genome 001 has one instance of the functional domain for code 1, zero instances of the functional domain for code 2, 3 instances of the functional domain for code 3, 5 instances of the functional domain for code 4, and so on.

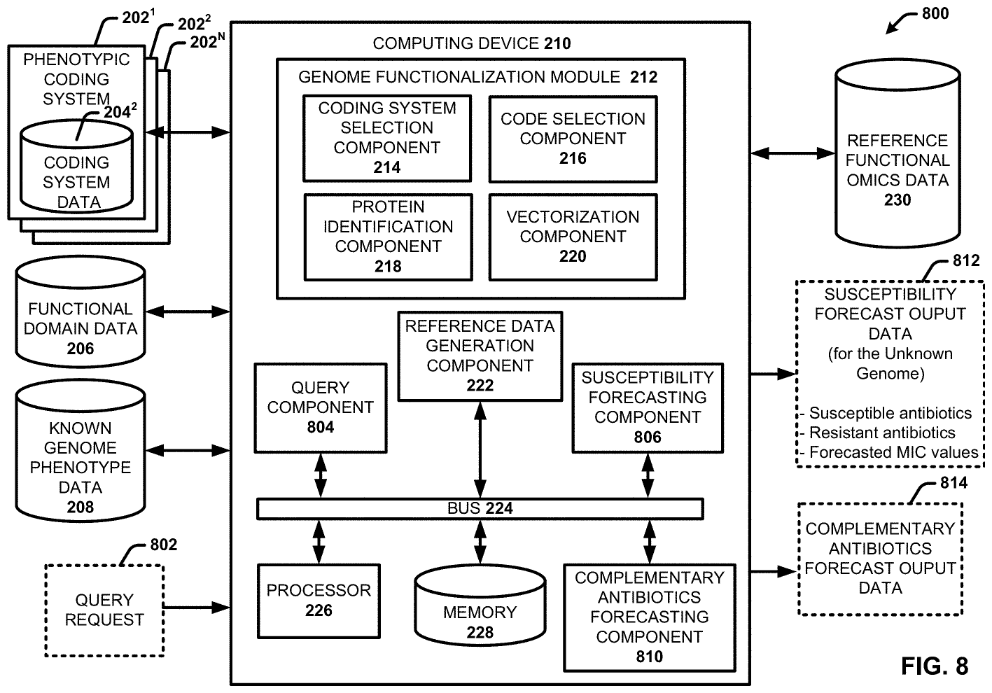

As can be seen in table 500, the FCVs for the six genomes vary, indicating the functional capacity of the respective genomes also varies. Correlations between the FCVs for the respective genomes and their AMR status can also be observed in Table 500. For instance, as can be seen in Table 500, the FCVs for the resistant genomes (e.g., Genomes001-003) all include lower values for codes 1-3 and higher values for codes 4-6 relative to the FCVs for the susceptible genomes (e.g., Genomes004-006). This indicates that genomes which exhibit low functional capacity of the features corresponding to codes 1-3 and higher functional capacity of the features corresponding to codes 4-6 are more likely to be resistant to B-lactam. As described in greater detail infra with reference to FIG. 8, reference functional omics data such as that shown in Table 500 can be used to generate inferences regarding antibiotic resistance of unknown genomes to the specific antibiotic compound (e.g., B-lactam) based on correlations between FCVs generated for the unknown genomes and the FCVs for the known genomes. For example, if an FCV generated for an unknown genome is more similar to those of the resistant genomes than the susceptible genomes, it can be assumed that the unknown genome is likely resistant to B-lactam.

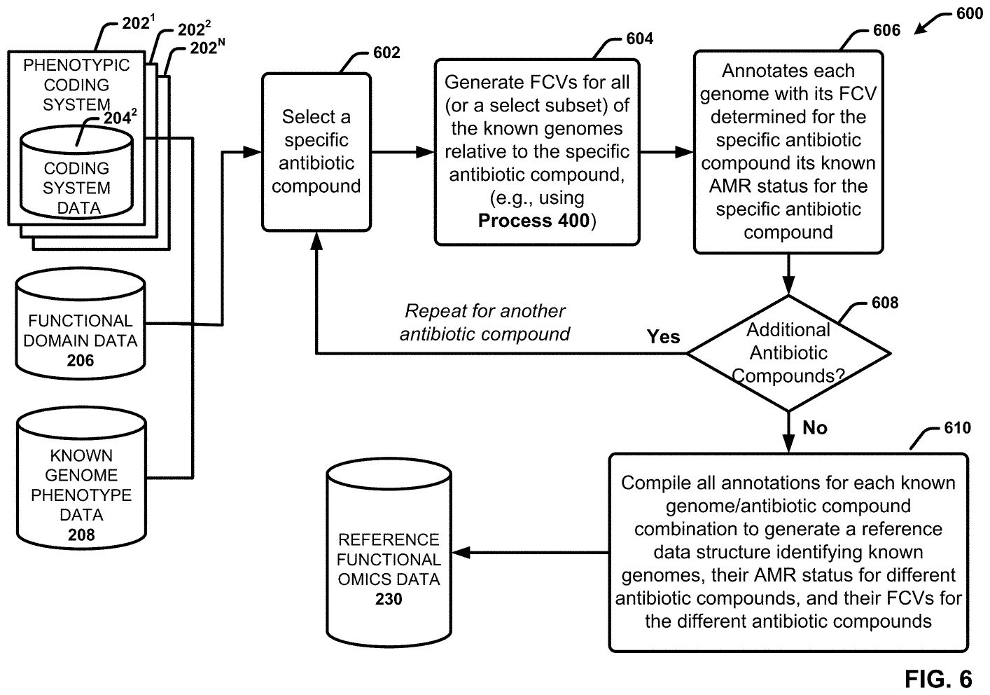

FIG. 6 illustrates a flow diagram of an example, non-limiting computer-implemented method 600 for generating reference functional omics that facilitates predicting antibiotic resistance and complementary combinations of antibiotics, in accordance with one or more embodiments described herein. Repetitive description of like elements employed in respective embodiments is omitted for sake of brevity.

Method 600 presents an example method for generating reference functional omics 230 data that reflects the AMR status of known genomes toward a plurality of different antibiotic compounds. In accordance with method 600, each FCV generated for a particular genome can be tailored to a single antibiotic compound. In this regard, each known genome can have a plurality of FCVs for different antibiotic compounds, wherein the different FCVs reflect the genomes functional capacity relative to the respective antibiotic compounds.

In this regard, at 602, the reference data generation component 222 can select a specific antibiotic compound for which the AMR status of known genomes is provided in the known genome phenotype data 208. At 604, the reference data generation component 222 can generate FCVs for all (or a select subset) of the known genomes relative to the specific antibiotic compound (e.g., using process 400). At 606, the reference data generation component 222 can annotate each genome with its FCV determined for the specific antibiotic compound and its known AMR status for the specific antibiotic compound. At 608, the reference data generation component 222 can determine whether AMR information for the known genomes for any additional antibiotic compounds is provided in the known genome phenotype data 208. If so, then the reference data generation component 222 can select another antibiotic compound and repeat processes 602-608.

Once all the antibiotic compounds for which the known genomes AMR status is provided in the known genome phenotype data 208 have been covered, then at 610, the reference data generation component 222 can compile all the annotations for each known genome/antibiotic compound combination to generate a reference data structure identifying known genomes, their AMR status for different antibiotic compounds, and their FCVs for the different antibiotic compounds.

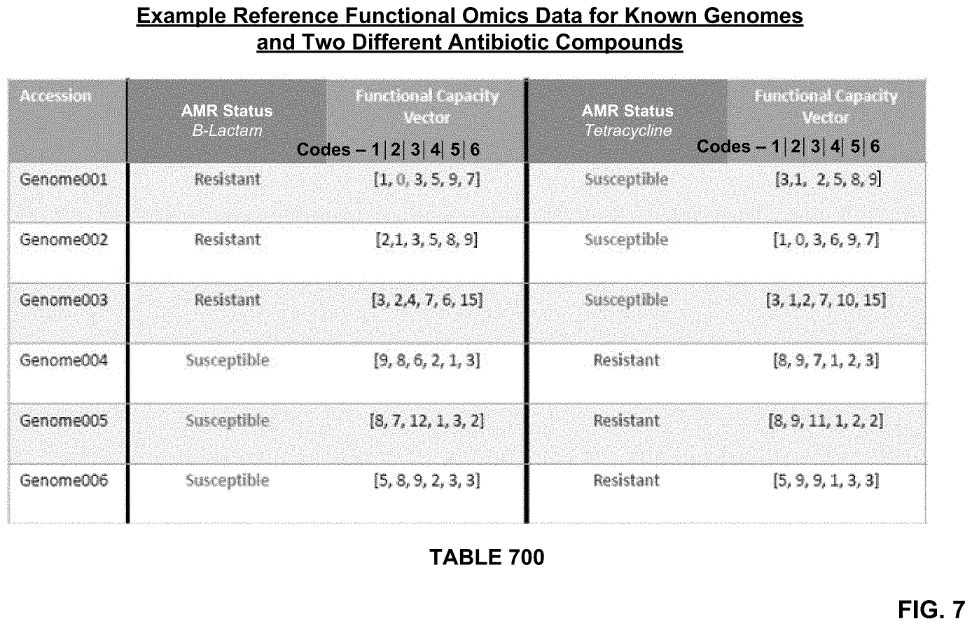

In this regard, FIG. 7 presents a table 700 comprising example reference functional omics data for known genomes and two different antibiotic compounds, in accordance with one or more embodiments. Table 700 provides example reference functional omics data 230 that can be generated using method 600. It should be appreciated that although table 700 depicts only two different antibiotic compounds, hundreds of different antibiotic compounds exist, and any number of different antibiotic compounds can be evaluated and annotated. As described in greater detail infra with reference to FIG. 8, reference functional omics data such as that shown in Table 700 can be used to generate inferences regarding complementary antibiotic combinations based on variances between the FCVs generated the unknown genomes for different antibiotic compounds.

FIG. 8 illustrates a block diagram of an example, non-limiting system 800 that facilitates predicting antibiotic resistance from functional omics data and recommending complementary combinations of antibiotics in accordance with one or more embodiments. System 800 includes same or similar features and functionalities as system 200 with the addition of query request 802, query component 804, susceptibility forecasting component 806, complementary antibiotics forecasting component 810, susceptibility forecast output data 812 and complementary antibiotics forecast output data 814. Repetitive description of like elements employed in respective embodiments is omitted for sake of brevity.

In one or more embodiments, the query component 804 can receive a query request that for one or more inferences based on the reference functional omics data 230. For example, as applied to antibiotic resistance prediction, in some embodiments, the query request 802 can identify an unknown bacterial genome whose AMR status relative to one or more antibiotic compounds is unknown. For instance, in some implementations, the unknown bacterial genome can include that of a bacterial organism infecting a patient. The query request 802 for an unknown bacterial genome can further include a request to receive information regarding susceptibility of the organism to one or more antibiotic compounds. For example, one implementation, the query request 802 can identify a specific antibiotic compound and include a request to determine information regarding whether the unknown genome is susceptible or resistant to the specific antibiotic compound. In another example implementation, the query request 802 can include a request to evaluate antibiotic resistance/susceptibility of the unknown genome relative many different antibiotic compounds. For example, some example query requests can ask questions including but not limited to: “What antibiotic compounds is this unknown genome susceptible to? “What MICs are needed for identified susceptible antibiotic compounds in order eradicate a patient's infection caused by the unknown organism?” What antibiotic compounds is this unknown genome resistant to?”, and “What degree of resistance or susceptibility does this unknown genome have toward various existing antibiotic compounds?”.

With these embodiments, the susceptibility forecasting component 806 can employ the genome functionalization module 212 to generate one or more FCVs for the unknown genome based on the query request in accordance with the techniques described herein (e.g., with reference to FIGS. 2-4). The susceptibility forecasting component 806 can then determine or infer the answer (or answers) to the query request 802 based on the one or more FCVs using the reference functional omics data 230. For example, the susceptibility forecasting component 806 can employ one or more statistical and/or machine learning techniques to determine the answer (or answers) to the query request based on correlations between the one or more FCVs generated for the unknown genome and reference FCVs for known genomes whose AMR status is known. With these embodiments, the reference functional omics data can include information identifying known genomes, their AMR statuses toward various known antibiotic compounds, and their FCVs relative to the different antibiotic compounds (e.g., as described with reference to FIGS. 5-7). The answer (or answers) to the query request in this context are referred to as susceptibility forecast output data 812. In various embodiments, the susceptibility forecast output data 812 can be presented to a user (e.g., via a device display or the like), and/or by the computing device 210 or another system for additional analysis.

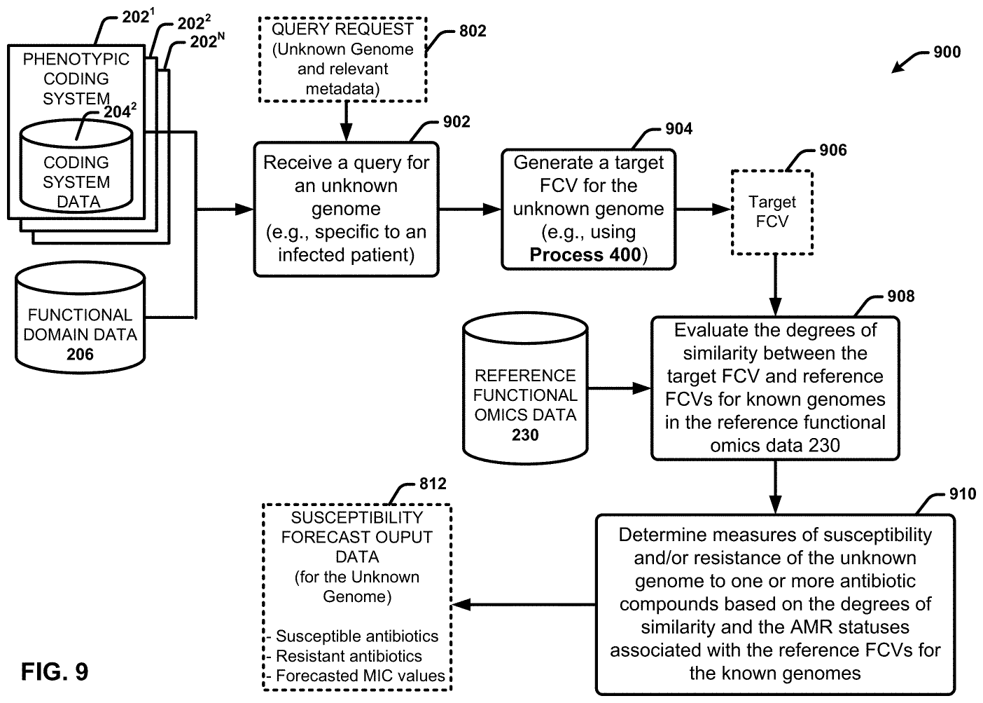

Various additional features and functionalities of the query component 804 and the susceptibility forecasting component 806 are now described with reference to FIGS. 9-12.

In this regard, FIG. 9 illustrates a flow diagram of an example, non-limiting computer-implemented method 900 for predicting antibiotic resistance from functional omics data and recommending complementary combinations of antibiotics in accordance with one or more embodiments. In various embodiments, method 900 can be performed by system 800 using the query component 804, the genome functionalization module 212 and the susceptibility forecasting component 806.

Method 900 can begin at 902 wherein the query component 804 receives a query request 802 for an unknown genome. For example, in accordance with method 900 the query request 802 can identify the genome of a bacterial organism infecting a patient and include a request to receive information regarding resistance and/or susceptibility of the organism to one or more antibiotic compounds. For instance, in one implementation, the query request 802 can identify a particular antibiotic compound (e.g., compound X) and request the susceptibility forecasting component 806 to determine whether and/or to what degree the unknown genome is resistant or susceptible to the particular antibiotic compound. In another example implementation, the query request 802 can identify the unknown genome and request susceptibility forecast output data 812 for the unknown genome that identifies one or more antibiotic compounds to which the unknown genome is expected to be susceptible to, one or more antibiotic compounds to which the unknown genome is expected to be resistant to and/or the forecasted MICs required for the susceptible antibiotic compounds for treating the patient.

In some implementations, the query request 802 can further include relevant metadata regarding the organism and/or the patient. For example, in some implementations, the metadata can identify or indicate the target features/functions (and/or corresponding codes as defined in the one or more phenotypic coding systems^{1-N}) to be used to generate an FCV for the organism. At 904, the genome functionalization module 212 can generate a target FCV 906 for the unknown genome (e.g., using process 400). In some embodiments, the target FCV 906 can be tailored to a specific antibiotic compound. With these embodiments, the genome functionalization module 212 can generate a plurality of different FCVs for the unknown genome, wherein each of the different FCVs are tailored to a particular antibiotic compound. In other embodiments, the target FCV 906 can generally reflect the genome's functional capacity relative to its antimicrobial resistance/susceptibility to a variety of different antibiotic compounds.

At 908, the susceptibility forecasting component 806 can evaluate the degrees of similarity (or differences) between the target FCV 906 and reference FCVs for known genomes in the reference functional omics data 230. At 910, the susceptibility forecasting component 806 can determine measures of susceptibility and/or resistance of the unknown genome to one or more antibiotic compounds based on the degrees of similarity and the AMR statuses associated with the reference FCVs for the known genomes (e.g., as provided by the reference functional omics data 230) to generate the susceptibility forecast output data 812.

For example, in one or more embodiments, at 906 the susceptibility forecasting component 806 can compare the target FCV 906 to FCVs determined for known genomes relative to a particular antibiotic compound whose AMR status for the particular antibiotic compound is known. At 910, the susceptibility forecasting component 806 can further determine or predict whether the unknown genome is susceptible or resistant to the antibiotic compound based on whether and/or to what degree the target FCV 906 is more similar to the FCVs of susceptible genomes or the FCVs of the resistant genomes. For example, in some embodiments, the susceptibility forecasting component 806 can classify the unknown genome as susceptible to a particular antibiotic compound if its degree of similarity to the susceptible genomes is greater than a threshold degree of similarly. Likewise, the susceptibility forecasting component 806 can classify the unknown genome as resistant to a particular antibiotic compound if its degree of similarity to the resistant genomes is greater than a threshold degree of similarly.

In some additional embodiments, the susceptibility forecasting component 806 can generate a susceptibility score for the unknown genome that reflects a degree of susceptibility or resistance of the unknown genome to a particular antibiotic compound based on how similar (or different) the unknown genome is to the susceptible genomes and/or the resistant genomes. With these embodiments, the susceptibility forecast output data 910 can also include the susceptibility score determined for the unknown genome that reflects its degree of susceptibility or resistance to the particular antibiotic compound.

Furthermore, in some implementations in which the AMR status provides the MICs for the antibiotic compounds, the susceptibility forecasting component 806 can also predict the MIC value for the particular antibiotic compound relative to the unknown genome based on the MIC values for the known genomes toward the particular antibiotic compound and the degree of similarity of the target FCV to the FCVs for the known genomes.

The susceptibility forecasting component 806 can perform this evaluation for not only a single antibiotic compound, but for many different antibiotic compounds to identify one or more antibiotic compounds that the unknown genome is expected to be susceptible to and/or one or more antibiotic compounds that the unknown genome is expected to be resistant to. In this regard, in the embodiment shown, the susceptibility forecast output data 812 can include information identifying one or more susceptible antibiotics to which the unknown genome is susceptible to, one or more resistant antibiotics to which the unknown genome is resistant to, and in some implementations, the forecasted MIC values (for the antibiotic compounds to which the unknown genome is susceptible to.

In some embodiments in which the susceptibility forecasting component 806 evaluates a plurality of different antibiotic compound (e.g., using process 900), the susceptibility forecasting component 806 can identify several different antibiotic compounds to which the unknown genome is susceptible toward and resistant toward. In some implementations of these embodiments, the susceptibility forecasting component 806 can further rank the identified antibiotic compounds based the degree of susceptibility or resistance of the unknown genome to the identified antibiotic compounds. For example, the susceptibility forecasting component 806 can rank the identified antibiotic compounds based from those which the unknown genome is considered most susceptible toward and those which the unknown genome is considered least susceptible toward. For example, in some implementations, the susceptibility forecasting component 806 can rank the evaluated antibiotic compounds based on their susceptibility scores and/or their forecasted MIC values.

The susceptibility forecasting component 806 can employ various statistical and/or machine learning techniques to evaluate the degrees of similarity between the target FCV 906 and reference FCVs for known genomes in the reference functional omics data 230. Some suitable machine learning algorithms/models that can be used by the susceptibility forecasting component 806 to evaluate the degrees of similarity between the target FCV 906 and reference FCVs for the known genomes in the reference functional omics data 230 can include but are not limited to: a nearest neighbor algorithm, a naïve Bayes algorithm, a decision tree algorithm, a boosting algorithm, a gradient boosting algorithm, a linear regression algorithm, a neural network algorithm, a clustering algorithm, a k-means clustering algorithm, an association rules algorithm, a q-learning algorithm, a temporal difference algorithm, a deep adversarial network algorithm, or a combination thereof.

For example, in one or more embodiments, the susceptibility forecasting component 806 can employ hierarchically clustering to evaluate the degrees of similarity (or differences) between the target FCV 906 and reference FCVs for known genomes in the reference functional omics data 230. With these embodiments, the susceptibility forecasting component 806 can generate a distance matrix that represents the distances between the reference FCVs and the target FCV 906.

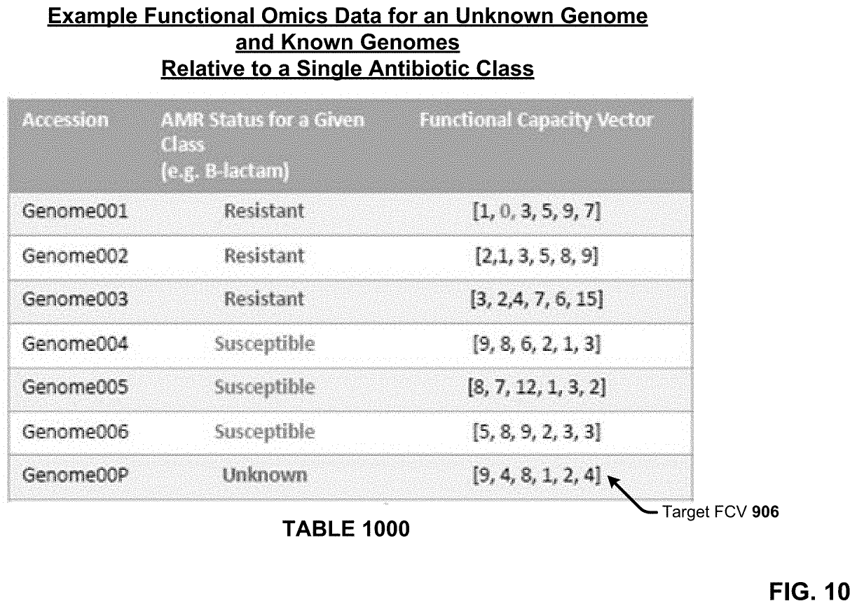

In this regard, with reference to FIG. 10, presented is a table 1000 comprising functional omics data for an unknown genome and known genomes relative to a single antibiotic class, in accordance with one or more embodiments. Table 1000 is the same as Table 500 with the addition of an unknown genome, identified as Genome00P and its FCV. In accordance with this example use case, the Genome00P corresponds to the genome of a bacterial organism infecting a patient whose AMR status to one or more antibiotic compounds (including at least B-lactam) is unknown. In this regard, the FCV for the unknown genome00P can correspond to the target FCV 906 generated in method 900.

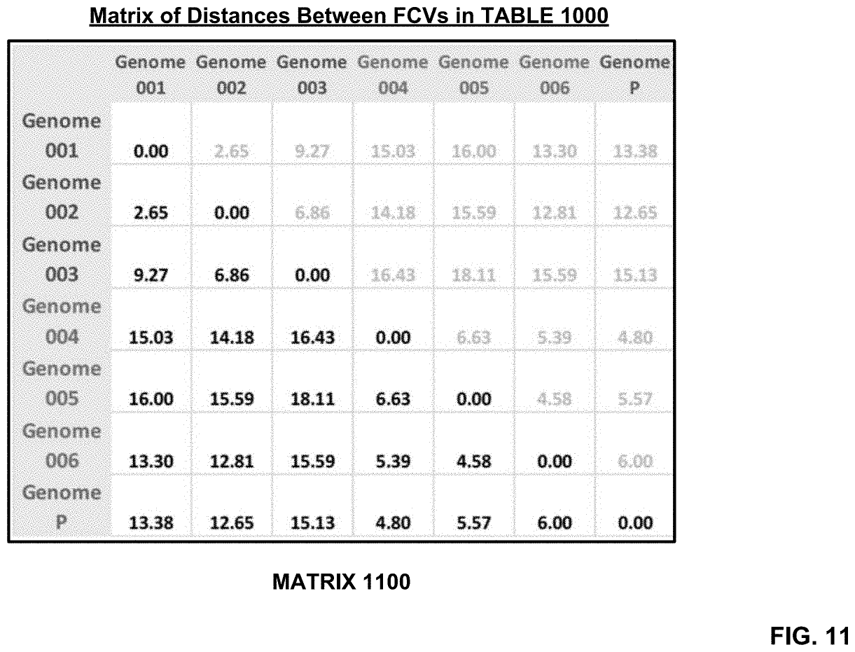

FIG. 11 illustrates an example matrix 1100 representing the distances between a functional capacity vector (e.g., FCV 906) for an unknown genome and the FCVs for known genomes, in accordance with one or more embodiments. In particular, matrix 1100 is a distance matrix representing the distances between the FCVs for Genomes 001-006 and Genome00P provided in Table 1000. In this regard, matrix 1100 reflects the degrees of similarity or differences between the target FCV 906 and FCVs for known genomes relative to a single antibiotic class/compound, B-lactam. In various embodiments, the susceptibility forecasting component 806 can determine or predict whether the Genome00P will be resistant or susceptible to B-lactam based on the distances between the FCV for the Genome00P (e.g., target FCV 906) and the FCVs for the resistant genomes (Genomes001-003) and the susceptible genomes (Genomes004-006). For example, in some implementations, the susceptibility forecasting component 806 can classify the unknown genome (Genome00P) as susceptible to B-lactam if its mean distance to the susceptible genomes is less than a threshold distance. Likewise, the susceptibility forecasting component 806 can classify the unknown genome (Genome00P) as resistant to B-lactam if its mean distance to the resistant genomes is less than a threshold distance. In another example implementation, the susceptibility forecasting component 806 can classify the unknown genome (Genome00P) as susceptible or resistant to an antibiotic compound reflected in a distance matrix such as matrix 1100 (e.g., which is B-lactam in the example shown) using the following Equation 1:

Δp=εₛ−εᵣ Equation 1,

wherein:
Δₚ=distance value for the unknown genome,
εₛ=minimum distance to a susceptible genome, and
εᵣ=minimum distance to a resistance genome.

In accordance with Equation 1, the susceptibility forecasting component 806 can determine a distance value (Δₚ) for the unknown genome based on the minimum distance to a susceptible genome (εₛ) minus the minimum distance to a resistant genome (εᵣ). The susceptibility forecasting component 806 can further employ a defined thresholding scheme that classifies the unknown genome as susceptible or resistant based on the distance value. For example, in some implementation, in accordance with Equation 1, the susceptibility forecasting component 806 can classify the unknown genome as susceptible if the distance value is a positive value and/or is greater than a defined threshold. Likewise, the susceptibility forecasting component 806 can classify the unknown genome as resistant if the distance value is a negative value and/or is absolute values is greater than a defined threshold.

The above embodiment can be used by susceptibility forecasting component 806 for single linkage clustering. However, in various embodiments, the susceptibility forecasting component 806 can also evaluate mixed annotations within a cluster of similar distances. With these embodiments, the susceptibility forecasting component 806 can employ more sophisticated machine learning methods (e.g., k-means clustering or the like) to evaluate the degrees of similarity (or differences) between a target for an unknown genome (e.g., target FCV 906) and reference FCVs for known genomes in the reference functional omics data 230.

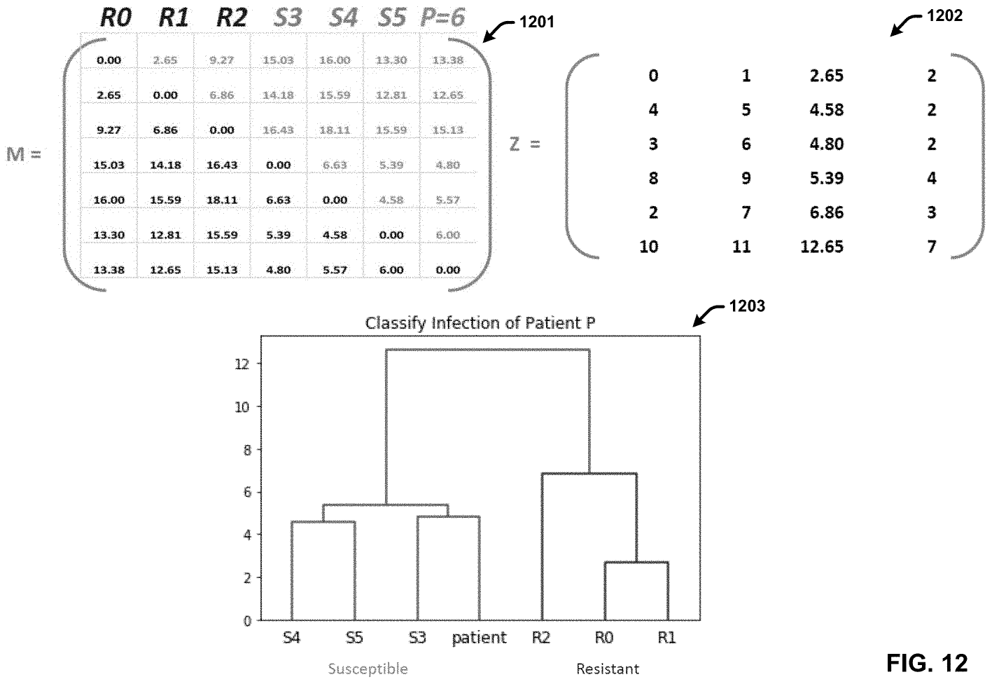

For example, FIG. 12 demonstrates an example of hierarchical (e.g., single linkage) clustering by FCVs for antibiotic resistance prediction, in accordance with one or more embodiments. In the embodiment shown, 1201 corresponds to the distance matrix 1100 (i.e., a matrix of distances (Δₚ)). Table 1202 presents the Z-scores that correspond to the output of single linkage clustering. The format of the returned linkage algorithm is a (n−1) by 4 matrix Z. At the i-th iteration, clusters with indices Z[i, 0] and Z[i, 1] are combined to form cluster n+i. A cluster with an index less than n corresponds to one of the original observations. The distance between clusters Z[i, 0] and Z[i, 1] is given by Z[i, 2]. The fourth value Z[i, 3] represents the number of original observations in the newly formed cluster.

Graph 1203 is a dendrogram constructed from the Z-scores in table 1202. In this regard Graph 1203 is derived from the pairwise distances between unknown genome FCV and the susceptible and resistant genomes as a function of the Z-scores and distances. In accordance with this example implementation, the susceptibility forecasting component 806 can classify the unknown genome as susceptible or resistant based on proximity to ground truth genomes (other genomes in the dendrogram). In the example the genome from a patient is found to cluster with ground truth susceptible genomes (and is, therefore, a susceptible genome).

With reference again to FIG. 8, in addition to employing the reference functional omics data 230 to generate susceptibility forecast output data 812 for new bacterial genomes whose AMR status is unknown, the computing device can also include complementary antibiotics forecasting component 810 to predict complementary antibiotic compound combinations that are expected to be more effective together than alone for treating certain bacterial infections. With these embodiments, the complementary antibiotics forecasting component 810 can employ one or more machine learning techniques to predict combinations of antibiotic compounds that are likely to be more effective together than alone for treating certain bacterial infections based on variations between FCVs and AMR statuses for different genomes when exposed to different antibiotic compounds. For example, in one or more embodiments, if for two different antibiotic compounds, the change in FCVs is in opposite directions for resistant and susceptible genomes, then those two antibiotic compounds can be expected to work better in combination. According to these embodiments, the complementary antibiotics forecasting component 810 can compare different combinations of antibiotics and evaluate the changes in the FCVs generated for different genomes' antibiotic response relative to their AMR status for the different antibiotic combinations to identify complementary antibiotic compound combinations. The complementary antibiotics forecasting component 810 can further generate complementary antibiotics forecast output data 814 regarding the identified complementary antibiotic compound combinations.

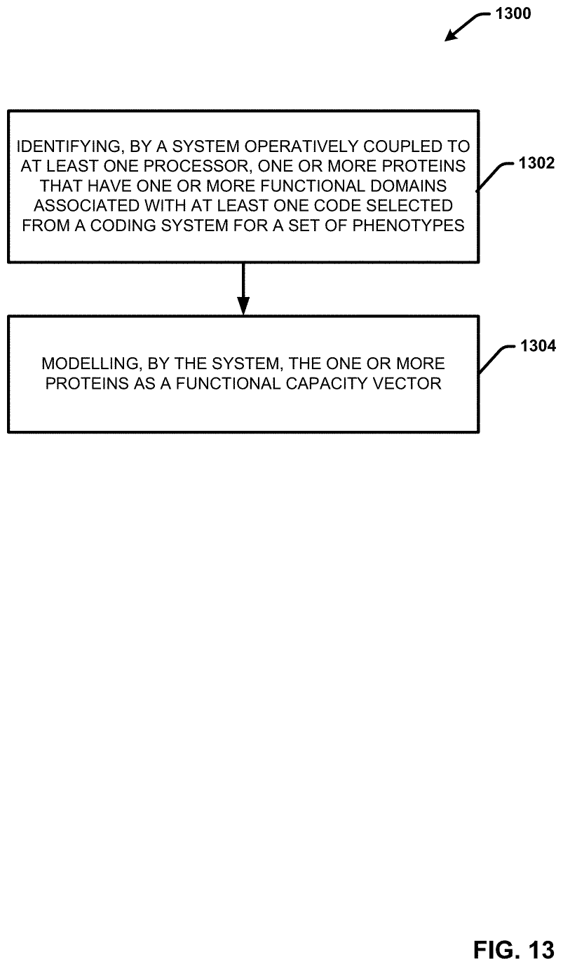

FIG. 13 illustrates a high-level flow diagram of an example, non-limiting computer-implemented method 1300 for identifying gene/protein sequences using dimensionally reduced coding vectors in accordance with one or more embodiments.

At 1302, method 1300 can comprise identifying (e.g., using protein identification component 218), by a system operatively coupled to a processor (e.g., system 200, system 800 and the like), one or more proteins that have one or more functional domains associated with at least one code selected from a coding system for a set of phenotypes (e.g., one or more of the phenotypic coding systems 202^{1-N}). At 1302, method 1300 can further comprise modelling, by the system, the one or more proteins as a functional capacity vector (FCV), (e.g., using vectorization component 220). In various embodiments, the FCV can indicate one or more first antibiotic compounds to which an organism within the set of phenotypes is resistant, one or more second antibiotic compounds to which an organism within the set of phenotypes is resistant, and/or one or more combinations of complementary antibiotic compounds.

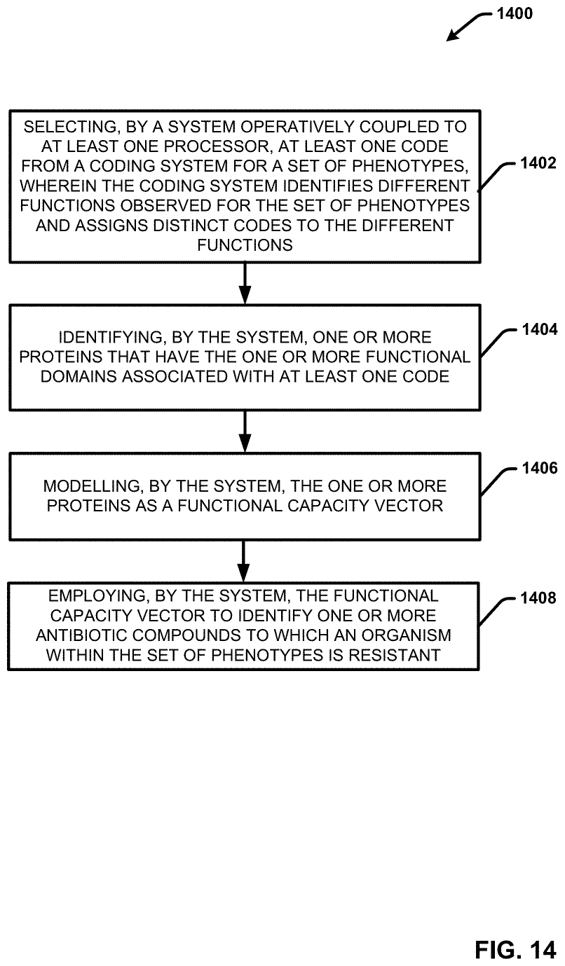

FIG. 14 illustrates a flow diagram of an example, non-limiting computer-implemented method 1400 for predicting antibiotic resistance from functional omics data in accordance with one or more embodiments.

At 1402, method 1400 can comprise selecting (e.g., using code selection component 216), by a system operatively coupled to a processor (e.g., system 200, system 800 and the like) at least one code from a coding system for a set of phenotypes (e.g., one or more of the phenotypic coding systems 202^{1-N}), wherein the coding system identifies different functions observed for the set of phenotypes and assigns distinct cods to the different functions. At 1404, method 1400 further comprises identifying, by the system, one or more proteins that have one or more functional domains associated with the at least one code (e.g., using protein identification component 218). At 1406, method 1400 can further comprise modelling, by the system, the one or more proteins as a functional capacity vector (FCV), (e.g., using vectorization component 220). At 1408, method 1400 can further comprise employing, by the system, the FCV to identify one or more antibiotic compounds to which an organism within the set of phenotypes is resistant (e.g., using susceptibility forecasting component 806).

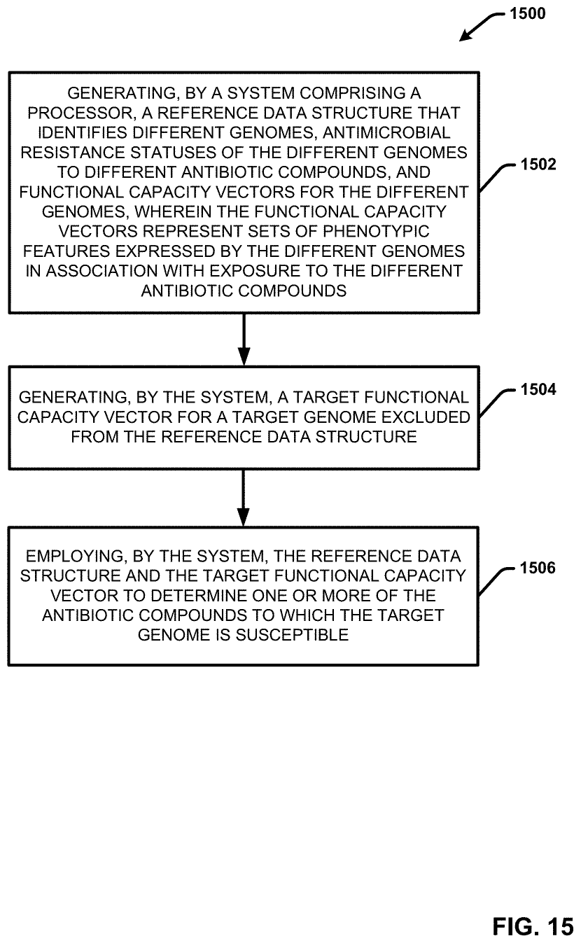

FIG. 15 illustrates a flow diagram of another example, non-limiting computer-implemented method 1500 for predicting antibiotic resistance from functional omics data in accordance with one or more embodiments.

At 1502, method 1500 can comprise generating, by a system comprising a processor (e.g., system 200, system 800 and the like), a reference data structure that identifies different genomes, antimicrobial resistance statuses of the different genomes to different antibiotic compounds, and functional capacity vectors for the different genomes (e.g., using reference data generation component 222), wherein the functional capacity vectors represent sets of phenotypic features expressed by the different genomes in association with exposure to the different antibiotic compounds. At 1504, method 1500 can further include generating, by the system, a target functional capacity vector (e.g., target FCV 906) for a target genome excluded from the reference data structure (e.g., by the using susceptibility forecasting component 806 using the genome functionalization module 212). At 1506, method 1500 can further comprise employing, by the system, the reference data structure and the target functional capacity vector to determine one or more of the antibiotic compounds to which the target genome is susceptible (e.g., using susceptibility forecasting component 806).

It should be noted that, for simplicity of explanation, in some circumstances the computer-implemented methodologies are depicted and described herein as a series of acts. It is to be understood and appreciated that the subject innovation is not limited by the acts illustrated and/or by the order of acts, for example acts can occur in various orders and/or concurrently, and with other acts not presented and described herein. Furthermore, not all illustrated acts can be required to implement the computer-implemented methodologies in accordance with the disclosed subject matter. In addition, those skilled in the art will understand and appreciate that the computer-implemented methodologies could alternatively be represented as a series of interrelated states via a state diagram or events. Additionally, it should be further appreciated that the computer-implemented methodologies disclosed hereinafter and throughout this specification are capable of being stored on an article of manufacture to facilitate transporting and transferring such computer-implemented methodologies to computers. The term article of manufacture, as used herein, is intended to encompass a computer program accessible from any computer-readable device or storage media.

FIG. 16 can provide a non-limiting context for the various aspects of the disclosed subject matter, intended to provide a general description of a suitable environment in which the various aspects of the disclosed subject matter can be implemented. FIG. 16 illustrates a block diagram of an example, non-limiting operating environment in which one or more embodiments described herein can be facilitated. Repetitive description of like elements employed in other embodiments described herein is omitted for sake of brevity.

With reference to FIG. 16, a suitable operating environment 1600 for implementing various aspects of this disclosure can also include a computer 1612. The computer 1612 can also include a processing unit 1614, a system memory 1616, and a system bus 1618. The system bus 1618 couples system components including, but not limited to, the system memory 1616 to the processing unit 1614. The processing unit 1614 can be any of various available processors. Dual microprocessors and other multiprocessor architectures also can be employed as the processing unit 1614. The system bus 1618 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MCA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1394), and Small Computer Systems Interface (SCSI).

The system memory 1616 can also include volatile memory 1620 and nonvolatile memory 1622. The basic input/output system (BIOS), containing the basic routines to transfer information between elements within the computer 1612, such as during start-up, is stored in nonvolatile memory 1622. Computer 1612 can also include removable/non-removable, volatile/non-volatile computer storage media. FIG. 16 illustrates, for example, a disk storage 1624. Disk storage 1624 can also include, but is not limited to, devices like a magnetic disk drive, floppy disk drive, tape drive, Jaz drive, Zip drive, LS-100 drive, flash memory card, or memory stick. The disk storage 1624 also can include storage media separately or in combination with other storage media. To facilitate connection of the disk storage 1624 to the system bus 1618, a removable or non-removable interface is typically used, such as interface 1626. FIG. 16 also depicts software that acts as an intermediary between users and the basic computer resources described in the suitable operating environment 1600. Such software can also include, for example, an operating system 1628. Operating system 1628, which can be stored on disk storage 1624, acts to control and allocate resources of the computer 1612.

System applications 1630 take advantage of the management of resources by operating system 1628 through program modules 1632 and program data 1634, e.g., stored either in system memory 1616 or on disk storage 1624. It is to be appreciated that this disclosure can be implemented with various operating systems or combinations of operating systems. A user enters commands or information into the computer 1612 through input device(s) 1636. Input devices 1636 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1614 through the system bus 1618 via interface port(s) 1638. Interface port(s) 1638 include, for example, a serial port, a parallel port, a game port, and a universal serial bus (USB). Output device(s) 1640 use some of the same type of ports as input device(s) 1636. Thus, for example, a USB port can be used to provide input to computer 1612, and to output information from computer 1612 to an output device 1640. Output adapter 1642 is provided to illustrate that there are some output devices 1640 like monitors, speakers, and printers, among other output devices 1640, which require special adapters. The output adapters 1642 include, by way of illustration and not limitation, video and sound cards that provide a means of connection between the output device 1640 and the system bus 1618. It should be noted that other devices and/or systems of devices provide both input and output capabilities such as remote computer(s) 1644.

Computer 1612 can operate in a networked environment using logical connections to one or more remote computers, such as remote computer(s) 1644. The remote computer(s) 1644 can be a computer, a server, a router, a network PC, a workstation, a microprocessor based appliance, a peer device or other common network node and the like, and typically can also include many or all of the elements described relative to computer 1612. For purposes of brevity, only a memory storage device 1646 is illustrated with remote computer(s) 1644. Remote computer(s) 1644 is logically connected to computer 1612 through a network interface 1648 and then physically connected via communication connection 1650. Network interface 1648 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 1650 refers to the hardware/software employed to connect the network interface 1648 to the system bus 1618. While communication connection 1650 is shown for illustrative clarity inside computer 1612, it can also be external to computer 1612. The hardware/software for connection to the network interface 1648 can also include, for exemplary purposes only, internal and external technologies such as, modems including regular telephone grade modems, cable modems and DSL modems, ISDN adapters, and Ethernet cards.

One or more embodiments described herein can be a system, a method, an apparatus and/or a computer program product at any possible technical detail level of integration. The computer program product can include a computer readable storage medium (or media) having computer readable program instructions thereon for causing a processor to carry out aspects of one or more embodiment. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. In this regard, in various embodiments, a computer readable storage medium as used herein can include non-transitory and tangible computer readable storage mediums.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of one or more embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of one or more embodiments.

Aspects of one or more embodiments are described herein with reference to flowchart illustrations and/or block diagrams of methods, apparatus (systems), and computer program products according to embodiments. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and other devices to function in a particular manner, such that the computer readable storage medium having instructions stored therein comprises an article of manufacture including instructions which implement aspects of the function/act specified in the flowchart and block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and block diagram block or blocks.

The flowchart and block diagrams in the Figures illustrate the architecture, functionality, and operation of possible implementations of systems, methods, and computer program products according to various embodiments described herein. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and flowchart illustration, and combinations of blocks in the block diagrams and flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on one or more computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices. For example, in one or more embodiments, computer executable components can be executed from memory that can include or be comprised of one or more distributed memory units. As used herein, the term “memory” and “memory unit” are interchangeable. Further, one or more embodiments described herein can execute code of the computer executable components in a distributed manner, e.g., multiple processors combining or working cooperatively to execute code from one or more distributed memory units. As used herein, the term “memory” can encompass a single memory or memory unit at one location or multiple memories or memory units at one or more locations.

As used in this application, the terms “component,” “system,” “platform,” “interface,” and the like, can refer to and can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and a computer. By way of illustration, both an application running on a server and the server can be a component. One or more components can reside within a process or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that can provide specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

The term “facilitate” as used herein is in the context of a system, device or component “facilitating” one or more actions or operations, in respect of the nature of complex computing environments in which multiple components and/or multiple devices can be involved in some computing operations. Non-limiting examples of actions that may or may not involve multiple components and/or multiple devices comprise transmitting or receiving data, establishing a connection between devices, determining intermediate results toward obtaining a result (e.g., including employing machine learning and artificial intelligence to determine the intermediate results), etc. In this regard, a computing device or component can facilitate an operation by playing any part in accomplishing the operation. When operations of a component are described herein, it is thus to be understood that where the operations are described as facilitated by the component, the operations can be optionally completed with the cooperation of one or more other computing devices or components, such as, but not limited to: sensors, antennae, audio and/or visual output devices, other devices, etc.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches, and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
identifying, by a system operatively coupled to at least one processor, one or more proteins that have one or more functional domains associated with at least one code selected from a coding system for a set of phenotypes; and
modelling, by the system, the one or more proteins as a functional capacity vector.

2. The method of claim 1, further comprising:
selecting, by the system, the coding system based on a phenotype of interest.

3. The method of claim 1, further comprising:
applying, by the system, one or more restrictions for the coding system restrictions in association with the selecting.

4. The method of claim 1, further comprising:
selecting, by the system, the at least one code based on a phenotype of interest.

5. The method of claim 1, further comprising:
employing, by the system, the functional capacity vector to identify one or more antibiotic compounds to which an organism within the set of phenotypes is resistant.

6. The method of claim 1, further comprising:
employing, by the system, the functional capacity vector to identify one or more antibiotic compounds to which an organism within the set of phenotypes is susceptible.

7. The method of claim 1, further comprising:
employing, by the system, the functional capacity vector to identify one or more antibiotic compound combinations to which an organism within the set of phenotypes is susceptible.

8. The method of claim 1, further comprising:
employing, by the system, the functional capacity vector to predict one or more minimum inhibitory concentrations for one or more antibiotic compounds against an organism within the set of phenotypes.

9. The method of claim 1, further comprising:
employing, by the system, the functional capacity vector to predict one or more minimum inhibitory concentrations for one or more antibiotic compound combinations against an organism within the set of phenotypes.

10. A system, comprising:
a memory that stores computer executable components;
a processor that executes the computer executable components stored in the memory, wherein the computer executable components comprise:
a protein identification component that identifies one or more proteins that have one or more functional domains associated with at least one code selected from a coding system for a set of phenotypes; and
a vectorization component that models the one or more proteins as a functional capacity vector.

11. The system of claim 10, wherein the computer executable components further comprise:
a coding system selection component that that selects the coding system based on a phenotype of interest.

12. The system of claim 10, wherein the computer executable components further comprise:
a code selection component that that selects the at least one code based on a phenotype of interest.

13. The system of claim 10, wherein the computer executable components further comprise:
a susceptibility forecasting component that employs the functional capacity vector to identify one or more antibiotic compounds to which an organism within the set of phenotypes is resistant.

14. The system of claim 10, wherein the computer executable components further comprise:
a susceptibility forecasting component that employs the functional capacity vector to identify one or more antibiotic compounds to which an organism within the set of phenotypes is susceptible.

15. The system of claim 10, wherein the computer executable components further comprise:
a susceptibility forecasting component that employs pairwise distances between functional capacity vectors to perform hierarchical or clustering or k-mean clustering to identify one or more antibiotic compounds to which an organism within the set of phenotypes is susceptible.

16. The system of claim 10, wherein the computer executable components further comprise:
a susceptibility forecasting component that employs the functional capacity vector to predict one or more minimum inhibitory concentrations for one or more antibiotic compounds against an organism included within the set of phenotypes.

17. The system of claim 10, wherein the computer executable components further comprise:
a combination forecasting component that employs the functional capacity vector to identify one or more antibiotic compound combinations to which an organism within the set of phenotypes is susceptible.

18. A computer program product for representing a genome with a dimensionally reduced coding vector that represents one or more target functions associated with the genome within a target phenotypic space the computer program product comprising a computer readable storage medium having program instructions embodied therewith, the program instructions executable by a processing component to cause the processing component to:
identify one or more target genes of the genome that encode one or more proteins responsible for the one or more target functions; and
generate a functional capacity vector for the genome using one or more distinct codes assigned to the one or more target functions.

19. The computer program product of claim 18, wherein the program instructions further cause the processing component to:
select at least one coding system for a set of phenotypes included in the target phenotypic space, wherein the at least one coding system identifies different functions observed for the set of phenotypes and assigns distinct codes to the different functions; and
determine the one or more distinct codes using the at least one coding system.

20. The computer program product of claim 18, wherein the program instructions further cause the processing component to:
determine one or more functional domains respectively associated with the one or more distinct codes;
identify the one or more proteins based on the one or more proteins comprising the one or more functional domains;
generate the functional capacity vector based on the one or more proteins; and
employ the functional capacity vector to identify one or more antibiotic compounds to which an organism included within target phenotypic space is susceptible.

21. A method comprising:
generating, by a system comprising a processor, a reference data structure that identifies different genomes, antimicrobial resistance statuses of the different genomes to different antibiotic compounds, and functional capacity vectors for the different genomes, wherein the functional capacity vectors represent sets of phenotypic features expressed by the different genomes in association with exposure to the different antibiotic compounds;
generating, by the system, a target functional capacity vector for a target genome excluded from the reference data structure; and
employing, by the system, the reference data structure and the target functional capacity vector to determine one or more of the antibiotic compounds to which the target genome is susceptible.

22. The method of claim 21, wherein the employing comprises employing one or more machine learning algorithms to facilitate identifying the one or more antibiotic compounds based on degrees of similarity between the target functional capacity vector and the functional capacity vectors.

23. The method of claim 20, wherein the antimicrobial statuses of the different genomes comprise minimum inhibitory concentration values, and wherein the method further comprises employing, by the system, the reference data structure and the target functional capacity vector to predict one or more minimum inhibitory concentration values for one or more of the antibiotic compounds against the target genome.

24. A system, comprising:
a memory that stores computer executable components;
a processor that executes the computer executable components stored in the memory, wherein the computer executable components comprise:
a reference data generation component that generates a reference data structure identifying different genomes, antimicrobial resistance statuses of the different genomes to different antibiotic compounds, and functional capacity vectors for the different genomes, wherein the functional capacity vectors represent sets of phenotypic features expressed by the different genomes in association with exposure to the different antibiotic compounds;
a vectorization component that generates a target functional capacity vector for a target genome excluded from the reference data structure; and
a susceptibility forecasting component that employs the reference data structure and the target functional capacity vector to determine one or more of the antibiotic compounds to which the target genome is susceptible.

25. The system of claim 24, wherein the susceptibility forecasting component employs one or more machine learning algorithms to facilitate determining the one or more antibiotic compounds based on degrees of similarity between the target functional capacity vector and the functional capacity vectors.
